(19) United States
(12) Patent Application Publication
(10) Pub. No.: US 2008/0249077 A1
(43) Pub. Date: Oct. 9, 2008

(54) 17.Beta.-Fluoromethoxycarbonyl-Androst-4-En-3-One Compounds With a 17.Alpha.-Carbonate Sustituent

(76) Inventors: Keith Biggadike, Hertfordshire (GB); Deborah Needham, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/093,613

(22) PCT Filed: Nov. 13, 2006
(86) PCT No.: PCT/EP2006/010894

§ 371 Date: May 14, 2008

(30) Foreign Application Priority Data
Nov. 15, 2005 (GB) 0523251.7

(51) Int. Cl. A61K 31/56; C07J 3/00; A61P 29/00; A61P 37/08

(52) U.S. Cl. 514/178; 552/610

(57) ABSTRACT

The present invention is directed to compounds of formula (I):

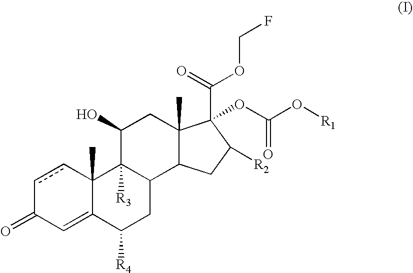

wherein
R₁ represents C₄-C₇ branched chain alkyl,
C₃-C₈ cycloalkyl optionally substituted by one or more groups independently selected from C₁-C₃ alkyl and methoxy,
C₄-C₆ cycloalkylmethyl wherein the methyl group is optionally substituted by a group selected from methyl or ethyl, or a bicycloalkyl group optionally substituted by one or more methyl groups;
R₂ represents hydrogen, a methyl group, which may be in either the a or D configuration, or a methylene group;
R₃ and R₄ are the same or a different group and each independently represents hydrogen, halogen or a methyl group;
and [symbol] represents a single or a double bond;
physiologically acceptable solvates thereof, pharmaceutical compositions thereof, methods of treatment using such compounds, and processes for preparing such compounds.

The present invention relates to compounds which are glucocorticoid receptor agonists of the androstane series and to processes for their preparation. The present invention also relates to pharmaceutical formulations containing the compounds and to therapeutic uses thereof, particularly for the treatment of inflammatory and allergic conditions.

Glucocorticosteroids which have anti-inflammatory properties are known and are widely used for the treatment of inflammatory disorders or diseases such as asthma and rhinitis. Androstane 17α-carbonate compounds said to have anti-inflammatory activity are disclosed in U.S. Pat. No. 4,996,335. Drugs of Today 2000, 36(5), 313-320, discloses loteprednol etabonate for the treatment of allergic diseases of the airways. We have identified a novel series of androstane 17α-carbonate derivatives. Thus, according to one aspect of the invention, there is provided a compound of formula (I)

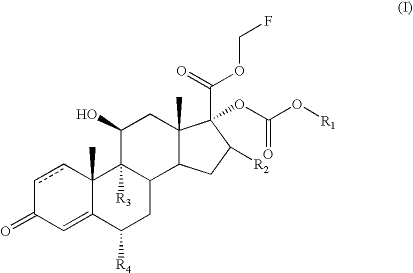

wherein
R₁ represents C₄-C₇ branched chain alkyl,
C₃-C₈ cycloalkyl optionally substituted by one or more groups independently selected from C₁-C₃ alkyl and methoxy,
C₄-C₆ cycloalkylmethyl optionally substituted by one or more groups selected from methyl or ethyl,
or a bicycloalkyl group optionally substituted by one or more methyl groups;
R₂ represents hydrogen, a methyl group, which may be in either the α or β configuration, or a methylene group;
R₃ and R₄ are the same or a different group and each independently represents hydrogen, halogen or a methyl group;
and [symbol] represents a single or a double bond;
or a physiologically acceptable solvate thereof.

Examples of solvates include hydrates.

References hereinafter to a compound according to the invention includes both compounds of formula (I) and solvates thereof.

In one embodiment R₁ represents C₄-C₆ branched chain alkyl.

Examples of C₄-C₆ branched alkyl groups which R₁ may represent include a 1,1-dimethylethyl, 1,1-dimethylpropyl, 2-ethylbutyl, 1-ethyl-2-methylpropyl, 1,2-dimethylpropyl or a 1,2,2-trimethylpropyl Isomer A group.

In one embodiment R₁ represents cyclohexyl optionally substituted by one or more groups independently selected from C₁-C₃ alkyl and methoxy.

In a further embodiment R₁ represents cyclohexyl optionally substituted by one or more groups independently selected from methyl and methoxy.

Examples of cyclohexyl groups which R₁ may represent include a (1R,2R)-2-(methyloxy)cyclohexyl, (1S,2S)-2-(methyloxy)cyclohexyl or a 3,3-dimethylcyclohexyl Isomer A group.

In one embodiment R₁ represents cyclopentylmethyl wherein the methyl group is optionally substituted by a group selected from methyl or ethyl.

Examples of optionally substituted cyclopentylmethyl groups which R₁ may represent include a cyclopentylmethyl or a 1-cyclopentylethyl Isomer A group.

In one embodiment R₁ represents a bicycloalkyl group optionally substituted by one or more methyl groups.

Examples of bicycloalkyl groups which R₁ may represent include 1RS,2RS,4SR-bicyclo[2.2.1]hept-2-yl Isomer B, 1RS,2SR,4SR bicyclo[2.2.1]hept-2-yl or a (1R,2R, 4S)-3,3-dimethylbicyclo[2.2.1]hept-2-yl group.

In one embodiment R₂ represents a methyl group. In a further embodiment R₂ represents methyl in the α-configuration.

In one embodiment R₃ and R₄, which can be the same or different, each represents hydrogen, methyl, fluorine or chlorine, for example hydrogen or fluorine. In one embodiment R₃ and R₄ are both fluorine.

In one embodiment [symbol] represents a double bond.

It is to be understood that the present invention covers all combinations of groups referred to hereinabove.

Compounds of formula (I) include:
Fluoromethyl(6α,11β,16α,17α)-17-({[(1,1-dimethylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylate;
Fluoromethyl(6α,11β,16α,17α)-17-({[(1-ethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[2-methyl-1-(1-methylethyl)propyl]oxy}carbonyl)oxy]-3-oxoandrosta-1,4-diene-17-carboxylate;
Fluoromethyl(6α,11β,16α,17α)-17-({[(2-ethylbutyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(2,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1S,2R)-2-methylcyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1R,2S)-2-methylcyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[4-(1-methylethyl)cyclohexyl]oxy}carbonyl)oxy]-3-oxoandrosta-1,4-diene-17-carboxylate;
Fluoromethyl(6α,11β,16α,17α)-17-({[(1S,2S,4R)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1R,2R,4S)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-{[(cycloheptyloxy)carbonyl]oxy}-6,9-difluoro-11-hydroxy-16-methyl-3-oxoandrosta-1,4-diene-17-carboxylate;
Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-{[(cyclooctyloxy)carbonyl]oxy}-6,9-difluoro-11-hydroxy-16-methyl-3-oxoandrosta-1,4-diene-17-carboxylate;
Fluoromethyl(6α,11β,16α,17α)-17-[({[(1S,3R,5S)-3,5-dimethylcyclohexyl]oxy}carbonyl)oxy]-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-3-oxo-17-({[(1-propylbutyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylate;

Fluoromethyl(6α,11β,16α,17α)-6,9-difluoro-11-hydroxy-16-methyl-17-[({[1-(1-methylethyl)butyl]oxy}carbonyl)oxy]-3-oxoandrosta-1,4-diene-17-carboxylate;
Fluoromethyl(6α,11β,16α,17α)-6,9-difluoro-11-hydroxy-16-methyl-3-oxo-17-({[(2,2,3,3-tetramethylcyclopropyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-[({[(1S,2R,4S)-1,7,7-trimethylbicyclo[2.2.1]hept-2-yl]oxy}carbonyl)oxy]androsta-1,4-diene-17-carboxylate;

Fluoromethyl(6α,11β,16α,17α)-6,9-difluoro-11-hydroxy-16-methyl-3-oxo-17-[({[(1R,2S,4R)-1,7,7-trimethylbicyclo[2.2.1]hept-2-yl]oxy}carbonyl)oxy]androsta-1,4-diene-17-carboxylate;

Fluoromethyl(6α,11β,16α,17α)-6,9-difluoro-11-hydroxy-16-methyl-3-oxo-17-[({[(1R,2R,4R)-1,7,7-trimethylbicyclo[2.2.1]hept-2-yl]oxy}carbonyl)oxy]androsta-1,4-diene-17-carboxylate;

Fluoromethyl(6α,11β,16α,17α)-6,9-difluoro-11-hydroxy-16-methyl-3-oxo-17-[({[(1S,2S,4S)-1,7,7-trimethylbicyclo[2.2.1]hept-2-yl]oxy}carbonyl)oxy]androsta-1,4-diene-17-carboxylate;
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate;

Fluoromethyl(6α,11β,16α,17α)-17-{[(cyclopentyloxy)carbonyl]oxy}-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylate;

Fluoromethyl(6α,11β,16α,17α)-6,9-difluoro-11-hydroxy-16-methyl-3-oxo-17-[({[(1R,2R,4S)-1,3,3-trimethylbicyclo[2.2.1]hept-2-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1R,2R,3R,5S)-2,6,6-trimethylbicyclo[3.1.1]hept-3-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1S,2S,3S,5R)-2,6,6-trimethylbicyclo[3.1.1]hept-3-yl]oxy}carbonyl)oxy]androsta-1,4-diene-17-carboxylate;
Fluoromethyl(6α,11β,16α,17α)-17-({[(cis-4-ethylcyclohexyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(trans-4-ethylcyclohexyl)oxy]carbonyl}oxy)-6,9-difluoro-11-hydroxy-16-methyl-3-oxoandrosta-1,4-diene-17-carboxylate; and
Fluoromethyl(6α,11β,16α,17α)-17-({[(1-ethyl-2,2-dimethylpropyl)oxy]carbonyl}oxy)-6,9-difluoro-11-hydroxy-16-methyl-3-oxoandrosta-1,4-diene-17-carboxylate.

In a further embodiment compounds of formula (I) include:
Fluoromethyl(6α,11β,16α,17α)-17-({[(1,1-dimethylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17≠)-17-({[(2-ethylbutyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylate Isomer B;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate Isomer A; and
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate.

In a further embodiment compounds include:
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylate Isomer B;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate Isomer A; and
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate.

The compounds of formula (I) have potentially beneficial anti-inflammatory or anti-allergic effects, particularly upon topical administration, demonstrated by, for example, their ability to bind to the glucocorticoid receptor and to illicit a response via that receptor. Hence, the compounds of formula (I) are potentially useful in the treatment of inflammatory and/or allergic disorders.

Examples of disease states in which the compounds of the invention may have utility include skin diseases such as eczema, psoriasis, allergic dermatitis neurodermatitis, pruritis and hypersensitivity reactions; inflammatory conditions of the nose, throat or lungs such as asthma (including allergen-induced asthmatic reactions), rhinitis (including hayfever), nasal polyps, chronic obstructive pulmonary disease, interstitial lung disease, and fibrosis; inflammatory bowel conditions such as ulcerative colitis and Crohn's disease; and auto-immune diseases such as rheumatoid arthritis.

Compounds of the invention may also have use in the treatment of conjunctiva and conjunctivitis.

It will be appreciated by those skilled in the art that reference herein to treatment extends to prophylaxis as well as the treatment of established conditions.

As mentioned above, compounds of formula (I) may be useful in human or veterinary medicine, in particular as anti-inflammatory and anti-allergic agents.

There is thus provided as a further aspect of the invention a compound of formula (I) or a physiologically acceptable solvate thereof for use in human or veterinary medicine, particularly in the treatment of patients with inflammatory and/or allergic conditions.

According to another aspect of the invention, there is provided the use of a compound of formula (I) or a physiologically acceptable solvate thereof for the manufacture of a medicament for the treatment of patients with inflammatory and/or allergic conditions.

In a further or alternative aspect, there is provided a method for the treatment of a human or animal subject with an inflammatory and/or allergic condition, which method comprises administering to said human or animal subject an effective amount of a compound of formula (I) or physiologically acceptable solvate thereof.

The compounds according to the invention may be formulated for administration in any convenient way, and the invention therefore also includes within its scope pharmaceutical compositions comprising a compound of formula (I) or physiologically acceptable solvate thereof together, if desirable, in admixture with one or more physiologically acceptable diluents or carriers.

Further, there is provided a process for the preparation of such pharmaceutical compositions which comprises mixing the ingredients.

The compounds according to the invention may, for example, be formulated for nasal, oral, buccal, sublingual, parenteral, local or rectal administration, especially local administration.

Local administration as used herein, includes administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (e.g. eye or nose drops), solutions/suspensions for nebulisation, suppositories, pessaries, retention enemas and chewable or suckable tablets or pellets (e.g. for the treatment of aphthous ulcers) or liposome or microencapsulation preparations.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Drops may be formulated with an aqueous or non-aqueous base also comprising one or more dispersing agents, solubilising agents, suspending agents or preservatives.

Spray compositions may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain a compound of formula (I) and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid, sorbitan trioleate or lecithin and cosolvents e.g. ethanol.

Advantageously, the formulations of the invention may be buffered by the addition of suitable buffering agents.

Powders for external application may be formed with the aid of any suitable powder base, for example, talc, lactose or starch. Suitable powders may be formulated with additional excipients, for example, cellobiose octo-acetate and magnesium stearate.

Capsules and cartridges for use in an inhaler or insufflator, of for example gelatine, may be formulated containing a powder mix for inhalation of a compound of the invention and a suitable powder base such as lactose or starch. Each capsule or cartridge may generally contain between 20 μg-10 mg of the compound of formula (I). Alternatively, the compound of the invention may be presented without excipients such as lactose.

The proportion of the active compound of formula (I) in the local compositions according to the invention depends on the precise type of formulation to be prepared but will generally be within the range of from 0.001 to 10% by weight. Generally, however for most types of preparations advantageously the proportion used will be within the range of from 0.005 to 1% and preferably 0.01 to 0.5%. However, in powders for inhalation or insufflation the proportion used will be within the range of from 0.1 to 5%.

Aerosol formulations are preferably arranged so that each metered dose or “puff” of aerosol contains 20 μg-2000 μg, preferably about 20 μg-500 μg of a compound of formula (I). Administration may be once daily or several times daily, for example 2, 3, 4 or 8 times, giving for example 1, 2 or 3 doses each time. The overall daily dose with an aerosol will be within the range 100 μg-10 mg preferably, 200 μg-2000 μg. The overall daily dose and the metered dose delivered by capsules and cartridges in an inhaler or insufflator will generally be double those with aerosol formulations.

Topical preparations may be administered by one or more applications per day to the affected area; over skin areas occlusive dressings may advantageously be used. Continuous or prolonged delivery may be achieved by an adhesive reservoir system.

For internal administration the compounds according to the invention may, for example, be formulated in conventional manner for oral, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms are, however, preferred as described below.

Preferred forms of preparation for internal administration are dosage unit forms i.e. tablets and capsules. Such dosage unit forms contain from 0.1 mg to 20 mg preferably from 2.5 to 10 mg of the compounds of the invention.

The compounds according to the invention may in general be given by internal administration in cases where systemic adreno-cortical therapy is indicated.

In general terms preparations, for internal administration may contain from 0.05 to 10% of the active ingredient dependent upon the type of preparation involved. The daily dose may vary from 0.1 mg to 60 mg, e.g. 5-30 mg, dependent on the condition being treated, and the duration of treatment desired.

Slow release or enteric coated formulations may be advantageous, particularly for the treatment of inflammatory bowel disorders.

The compound and pharmaceutical compositions according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents (e.g. antibiotics, antivirals), or antihistamines. The invention thus provides, in a further aspect, a combination comprising a compound of formula (I) or a pharmaceutically acceptable salt, solvate or physiologically functional derivative thereof together with one or more other therapeutically active agents, for example selected from an anti-inflammatory agent (for example another corticosteroid or an NSAID), an anticholinergic agent, a β₂-adrenoreceptor agonist, an antiinfective agent (e.g. an antibiotic or an antiviral), or an antihistamine. On embodiment of the invention encompasses combinations comprising a compound of formula (I) or a pharmaceutically acceptable solvate or physiologically functional derivative thereof together with a β₂-adrenoreceptor agonist, and/or an anticholinergic, and/or a PDE-4 inhibitor, and/or antihistamine.

One embodiment of the invention encompasses combinations comprising one or two other therapeutic agents.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

In one embodiment, the invention encompasses a combination comprising a compound of the invention together with a β₂-adrenoreceptor agonist

Examples of β₂-adrenoreceptor agonists include salmeterol (e.g. as racemate or a single enantiomer such as the R-enantiomer), salbutamol (e.g. as racemate or a single enantiomer such as the R-enantiomer), formoterol (e.g. as racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment the β₂-adrenoreceptor agonists are long-acting β₂-adrenoreceptor agonists, for example compounds which provide effective bronchodilation for about 12 hours or longer.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Other β₂-adrenoreceptor agonists include compounds of formula (XX):

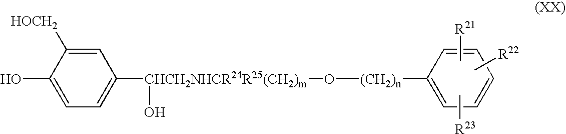

or a salt or solvate thereof, wherein:
m is an integer of from 2 to 8;
n is an integer of from 3 to 11,
with the proviso that m+n is 5 to 19,
R²¹ is —XSO₂NR²⁶R²⁷ wherein X is —(CH₂)ₚ— or C₂₋₆ alkenylene;
R²⁶ and R²⁷ are independently selected from hydrogen, C₁₋₆alkyl, C₃₋₇cycloalkyl, C(O)NR²⁸R²⁹, phenyl, and phenyl(C₁₋₄alkyl)-,
or R²⁶ and R²⁷, together with the nitrogen to which they are bonded, form a 5-, 6-, or 7-membered nitrogen containing ring, and R²⁶ and R²⁷ are each optionally substituted by one or two groups selected from halo, C₁₋₆alkyl, C₁₋₆haloalkyl, C₁₋₆alkoxy, hydroxy-substituted C₁₋₆alkoxy, —CO₂R²⁸, —SO₂NR²⁸R²⁹, —CONR²⁸R²⁹, —NR²⁸C(O)R²⁹, or a 5-, 6- or 7-membered heterocylic ring;
R²⁸ and R²⁹ are independently selected from hydrogen, C₁₋₆alkyl, C₃₋₆cycloalkyl, phenyl, and phenyl(C₁₋₄alkyl)-; and
p is an integer of from 0 to 6, preferably from 0 to 4;
R²² and R²³ are independently selected from hydrogen, C₁₋₆alkyl, C₁₋₆alkoxy, halo, phenyl, and C₁₋₆haloalkyl; and
R²⁴ and R²⁵ are independently selected from hydrogen and C₁₋₄alkyl with the proviso that the total number of carbon atoms in R²⁴ and R²⁵ is not more than 4.

Further examples of β₂-adrenoreceptor agonists include:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}-amino) heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Suitable corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. One embodiment of the invention encompasses corticosteroids including fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11-hydroxy-16α-methyl-3-oxo-androsta-1,4-diene-17β-carbothioic acid S-fluoromethyl ester.

Non-steroidal compounds having glucocorticoid agonism that may posess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis (eg. montelukast), iNOS inhibitors, tryptase and elastase inhibitors, beta-2 integrin antagonists and adenosine receptor agonists or antagonists (e.g. adenosine 2a agonists), cytokine antagonists (e.g. chemokine antagonists, such as a CCR3 antagonist) or inhibitors of cytokine synthesis, or 5-lipoxygenase inhibitors. In one embodiment an iNOS (inducible nitric oxide synthase inhibitor) is for oral administration. Examples of iNOS inhibitors include those disclosed in WO93/13055, WO98/30537, WO02/50021, WO95/34534 and WO99/62875. Examples CCR3 inhibitors include those disclosed in WO02/26722.

In one embodiment, the invention provides the use of the compounds of formula (I) in combination with a phosphodiesterase 4 (PDE4) inhibitor, for example in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds of interest include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds of interest include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further compounds of interest are disclosed in the published international patent applications WO04/024728 (Glaxo Group Ltd), PCT/EP2003/014867 (Glaxo Group Ltd) and PCT/EP2004/005494 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (e.g. as the bromide, CAS 22254-24-6, sold under the name Atrovent), oxitropium (e.g. as the bromide, CAS 30286-75-0) and tiotropium (e.g. as the bromide, CAS 136310-93-5, sold under the name Spiriva). Also of interest are revatropate (e.g. as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (e.g. as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other anticholinergic agents include compounds of formula (XXI), which are disclosed in U.S. patent application 60/487,981:

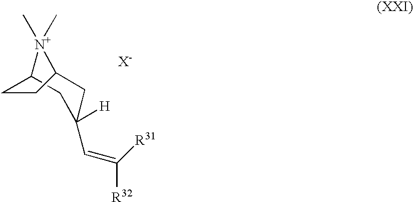

in which the preferred orientation of the alkyl chain attached to the tropane ring is endo;
R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;
X⁻ represents an anion associated with the positive charge of the N atom. X— may be but is not limited to chloride, bromide, iodide, sulfate, benzene sulfonate, and toluene sulfonate,
including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds of formula (XXII) or (XXIII), which are disclosed in U.S. patent application 60/511,009:

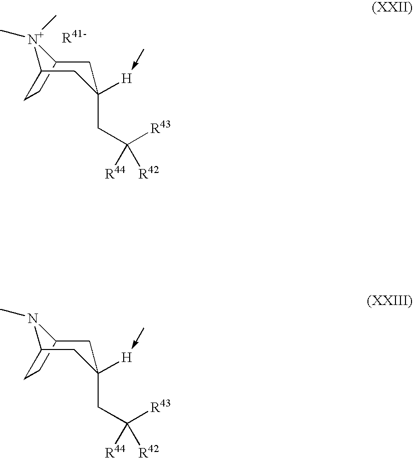

wherein:
the H atom indicated is in the exo position;
R⁴¹ represents an anion associated with the positive charge of the N atom. R⁴¹ may be but is not limited to chloride, bromide, iodide, sulfate, benzene sulfonate and toluene sulfonate;
R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having 6 to 10 carbon atoms), heterocycloalkyl (having 5 to 6 carbon atoms) and N or O as the heteroatom, heterocycloalkyl-alkyl (having 6 to 10 carbon atoms) and N or O as the heteroatom, aryl, optionally substituted aryl, heteroaryl, and optionally substituted heteroaryl;
R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), CH₂N(R⁴⁸)SO₂(R⁴⁶), —CH₂N(R⁴⁸)CO₂(R⁴⁵), —CH₂N(R⁴⁸)CONH(R⁴⁷);
R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;
R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;
R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl, including, for example:
(Endo)-3-(2-methoxy-2,2-di-thiophen-2-yl-ethyl)-8,8-dimethyl-8-azonia-bicyclo[3.2.1]octane iodide;
3-((Endo)-8-methyl-8-aza-bicyclo[3.2.1]oct-3-yl)-2,2-diphenyl-propionitrile;
(Endo)-8-methyl-3-(2,2,2-triphenyl-ethyl)-8-aza-bicyclo[3.2.1]octane;
3-((Endo)-8-methyl-8-aza-bicyclo[3.2.1]oct-3-yl)-2,2-diphenyl-propionamide;
3-((Endo)-8-methyl-8-aza-bicyclo[3.2.1]oct-3-yl)-2,2-diphenyl-propionic acid;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
3-((Endo)-8-methyl-8-aza-bicyclo[3.2.1]oct-3-yl)-2,2-diphenyl-propan-1-ol;
N-Benzyl-3-((endo)-8-methyl-8-aza-bicyclo[3.2.1]oct-3-yl)-2,2-diphenyl-propionamide;
(Endo)-3-(2-carbamoyl-2,2-diphenyl-ethyl)-8,8-dimethyl-8-azonia-bicyclo[3.2.1]octane iodide;
1-Benzyl-3-[3-((endo)-8-methyl-8-aza-bicyclo[3.2.1]oct-3-yl)-2,2-diphenyl-propyl]-urea;
1-Ethyl-3-[3-((endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-acetamide;
N-[3-((Endo)-8-methyl-8-aza-bicyclo[3.2.1]oct-3-yl)-2,2-diphenyl-propyl]-benzamide;
3-((Endo)-8-methyl-8-aza-bicyclo[3.2.1]oct-3-yl)-2,2-di-thiophen-2-yl-propionitrile;
(Endo)-3-(2-cyano-2,2-di-thiophen-2-yl-ethyl)-8,8-dimethyl-8-azonia-bicyclo[3.2.1]octane iodide;
N-[3-((Endo)-8-methyl-8-aza-bicyclo[3.2.1]oct-3-yl)-2,2-diphenyl-propyl]-benzenesulfonamide;
[3-((Endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-methanesulfonamide; and/or

(Endo)-3-{2,2-diphenyl-3-[(1-phenyl-methanoyl)-amino]-propyl}-8,8-dimethyl-8-azonia-bicyclo[3.2.1]octane bromide.

Further compounds include:
(Endo)-3-(2-methoxy-2,2-di-thiophen-2-yl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
(Endo)-3-(2-carbamoyl-2,2-diphenyl-ethyl)-8,8-dimethyl-8-azonia-bicyclo[3.2.1]octane iodide;
(Endo)-3-(2-cyano-2,2-di-thiophen-2-yl-ethyl)-8,8-dimethyl-8-azonia-bicyclo[3.2.1]octane iodide; and/or
(Endo)-3-{2,2-diphenyl-3-[(1-phenyl-methanoyl)-amino]-propyl}-8,8-dimethyl-8-azonia-bicyclo[3.2.1]octane bromide.

Examples of antihistamines (also referred to as H1-receptor antagonists) include any one or more of the numerous antagonists known which inhibit H1-receptors, and are safe for human use. First generation antagonists, include derivatives of ethanolamines, ethylenediamines, and alkylamines, e.g diphenylhydramine, pyrilamine, clemastine, chloropheniramine. Second generation antagonists, which are non-sedating, include loratidine, desloratidine, terfenadine, astemizole, acrivastine, azelastine, levocetirizine fexofenadine and cetirizine.

In one embodiment of the invention the anti-histamines include loratidine, desloratidine, fexofenadine and cetirizine.

Further examples include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I), or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of formula (I) a pharmaceutically acceptable salt, solvate or physiologically functional derivative thereof together with a PDE4 inhibitor.

The invention thus provides, in a further aspect, a combination comprising a compound of formula (I) a pharmaceutically acceptable salt, solvate or physiologically functional derivative thereof together with a β₂-adrenorecptor agonist.

The invention thus provides, in a further aspect, a combination comprising a compound of formula (I) a pharmaceutically acceptable salt, solvate or physiologically functional derivative thereof together with an anticholinergic.

The invention thus provides, in a further aspect, a combination comprising a compound of formula (I) a pharmaceutically acceptable salt, solvate or physiologically functional derivative thereof together with an antihistamine.

The invention thus provides, in a further aspect, a combination comprising a compound of formula (I) a pharmaceutically acceptable salt, solvate or physiologically functional derivative thereof together with a PDE4 inhibitor and a β₂-adrenoreceptor agonist.

The invention thus provides, in a further aspect, a combination comprising a compound of formula (I) a pharmaceutically acceptable salt, solvate or physiologically functional derivative thereof together with an anticholinergic and a PDE-4 inhibitor.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. Preferably the individual compounds of such combinations may be administered simultaneously in a combined pharmaceutical combination. Appropriate doses of known therapeutic agents will be readily appreciated by those skilled in the art.

The compounds of formula (I) and solvates thereof may be prepared by the methodology described hereinafter, constituting a further aspect of this invention.

A process according to the invention for preparing a compound of formula (I) comprises reaction of a carboxylic acid of formula (II);

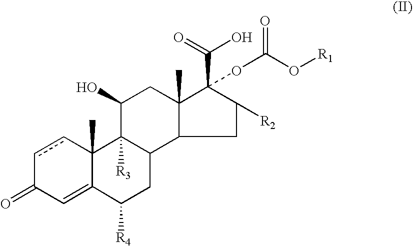

wherein R₁, R₂, R₃, R₄ and [symbol] are as defined above,
with a compound of formula L-CH₂—F wherein L represents a leaving group.

In this process the compound of formula (II) may be reacted with a compound of formula L-CH₂—F wherein L represents a leaving group such as halogen atom or a tosyl or mesyl group or the like, under standard conditions. For example the reaction may be performed in an inert polar organic solvent e.g. N,N-dimethylformamide in the presence of a base e.g. potassium carbonate, sodium carbonate.

Compounds of formula (II) may conveniently be employed as salts when such salts may be prepared in crystalline form, or as solvates.

Compounds of formula L-CH₂—F are either known or may be prepared by known methods.

Compounds of formula (II) may be prepared from the corresponding 17α-hydroxyl derivative of formula (III):

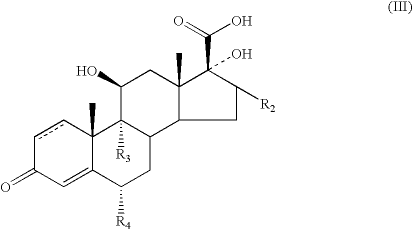

wherein R₂, R₃, R₄ and [symbol] are as defined above,
using for example, methodology similar to that described by G. H. Phillipps et al., to prepare 17α carboxylate esters (Journal of Medicinal Chemistry, (1994), 37, 3717-3729) and by Druzgala et al., to prepare the 17α carbonate ester loteprednol etabonate (Journal of Steroid Chemistry and Molecular Biology, (1991), 38, 149-154). The step typically comprises the reaction of the hydroxyacid (III) with a chloroformate R₁OCOCl in the presence of a mild base e.g. triethylamine in a suitable solvent e.g. dichloromethane. In the case of sterically encumbered R₁ groups anhydrides (R₁OCO)₂O may be preferred to the chloroformates.

Generally the chloroformate or anhydride would be employed in at least 2 times molar quantity relative to the compound of formula (III). The second mole of chloroformate or anhydride tends to react with the carboxylic acid moiety in the compound of formula (III) and would need to be removed by reaction with an amine such as diethylamine or 1-methylpiperazine. The chloroformates are either commercially available or are readily prepared by standard methodology e.g. by reaction of the corresponding alcohol R₁OH with phosgene or more preferably triphosgene in the presence of a base e.g. pyridine in a suitable solvent e.g. dichloromethane.

More conveniently, reaction of the 17α-hydroxyl derivative (III) with the chloroformate R₁OCOCl or anhydride (R₁OCO)₂O in pyridine solution often affords the 17α carbonate (II) directly.

Compounds of formula (III) are either known or may be prepared in accordance with procedures generally described by G. H. Phillipps et al., Journal of Medicinal Chemistry, (1994), 37, 3717-3729.

The following compounds of formula (II) are new and form an aspect of the invention:
(6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylic acid;
(6α,11β,16α,17α)-17-{[(cycloheptyloxy)carbonyl]oxy}-6,9-difluoro-11-hydroxy-16-methyl-3-oxoandrosta-1,4-diene-17-carboxylic acid;
(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylic acid;
(6α,11β,16α,17α)-17-{[(cyclooctyloxy)carbonyl]oxy}-6,9-difluoro-11-hydroxy-16-methyl-3-oxoandrosta-1,4-diene-17-carboxylic acid;
(6α,11β,16α,17α)-17-[({[(1S,3R,5S)-3,5-dimethylcyclohexyl]oxy}carbonyl)oxy]-6,9-difluoro-11-hydroxy-16-methyl-3-oxoandrosta-1,4-diene-17-carboxylic acid;
(6α,11β,16α,17α)-6,9-difluoro-11-hydroxy-16-methyl-17-[({[(1RS,2RS)-2-(methyloxy)cyclohexyl]oxy}carbonyl)oxy]-3-oxoandrosta-1,4-diene-17-carboxylic acid;
(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylic acid;
(6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylic acid;
(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylic acid;
(6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylic acid;
(6α,11β,16α,17α)-6,9-difluoro-11-hydroxy-16-methyl-3-oxo-17-({[(2,2,3,3-tetramethylcyclopropyl)oxy]carbonyl}oxy)androsta-1,4-diene-17-carboxylic acid;
(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylic acid;
(6α,11β,16α,17α)-6,9-difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylic acid; and
(6α,11β,16α,17α)-17-({[(1-ethyl-2,2-dimethylpropyl)oxy]carbonyl}oxy)-6,9-difluoro-11-hydroxy-16-methyl-3-oxoandrosta-1,4-diene-17-carboxylic acid.

Compounds of formula (I) and/or solvates thereof demonstrate agonism at the glucocorticoid receptor.

Compounds of formula (I) and/or solvates thereof may demonstrate good anti-inflammatory properties, with predictable pharmacokinetic and pharmacodynamic behaviour. They also may have an attractive side-effect profile, demonstrated, for example, by increased selectivity for the glucocorticoid receptor over the progesterone receptor and/or increased selectivity for glucocorticoid receptor mediated transrepression over transactivation and are likely to be compatible with a convenient regime of treatment in human patients.

The following non-limiting Examples illustrate the invention:

### EXAMPLES

### General

### Abbreviations

DMSO Dimethylsulphoxide
NMR Nuclear magnetic resonance
LCMS Liquid chromatography/mass spectrometry
MeCN Acetonitrile

Chromatographic purification was performed using pre-packed Bond Elut silica gel cartridges available commercially from Varian.

### NMR

¹H NMR spectra were recorded in DMSO-d₆ on a Bruker DPX 400 working at 400 MHz. The internal standard used was either tetramethylsilane or the residual protonated solvent at 2.50 ppm for DMSO-d₆.

### Mass Directed Autopreparative HPLC

Autopreparative HPLC was carried out using a Waters 600 gradient pump, Waters 2767 inject/collector, Waters Reagent Manager, Micromass ZMD mass spectrometer, Gilson Aspec waste collector and Gilson 115 post-fraction UV detector. The column used was typically a Supelco LCABZ++ column with dimension of 20 mm internal diameter by 100 mm in length. The stationary phase particle size is 5 μm. The flow rate was 20 ml/min and the runtime was 15 minutes, which comprises a 10-minute gradient followed by a 5 minute column flush and re-equilibration step.

Solvent A: Aqueous solvent=water+0.1% formic acid.

Solvent B: Organic solvent=MeCN:water 95:5+0.05% formic acid

Specific gradients used were dependent upon the retention time in the analytical system. For 1.5-2.2 min, 0-30% B, 2.0-2.8 min, 5-30% B, 2.5-3.0 min, 15-55% B, 2.8-4.0 min, 30-80% B and 3.8-5.5 min, 50-90% B.

### LCMS System

The LCMS system used was as follows:
Column: 3.3 cm×4.6 mm ID, 3 μm ABZ+PLUS from Supelco
Flow Rate: 3 ml/min
Injection Volume: 5 μl
Temp: RT
UV Detection Range: 215 to 330 nm
   Solvents: A: 0.1% Formic Acid+10 mMolar Ammonium Acetate.
B: 95% Acetonitrile+0.05% Formic Acid

| | Time | A % | B % |
|---|---|---|---|
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

### Intermediates

### Intermediate 1: (6α,11β,16α,17α)-17-({[(1,1-Dimethylethyl)oxy]carbonyl}oxy)-6,9-difluoro-11-hydroxy-16-methyl-3-oxoandrosta-1,4-diene-17-carboxylic acid

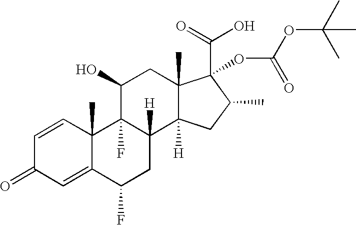

Bis(1,1-dimethylethyl) dicarbonate (121 mg, 0.56 mmol) was added to a stirred solution of (6α,11β,16α,17α)-6,9-difluoro-11,17-dihydroxy-16-methyl-3-oxoandrosta-1,4-diene-17-carboxylic acid (G. H. Phillipps et al., (1994) Journal of Medicinal Chemistry, 37, 3717-3729) (200 mg, 0.5 mmol) in pyridine (5 ml) and the mixture stirred at room temperature overnight. The solvent was evaporated in vacuo and the remaining residue stirred with 2M hydrochloric acid (20 ml). The resulting precipitate was collected by filtration, washed with water and dried in vacuo at 60° C. to give the title compound: LCMS retention time 3.27 min.

### Intermediate 2: (6α,11β,16α,17α)-17-({[(1,1-Dimethylpropyl)oxy]carbonyl}oxy)-6,9-difluoro-11-hydroxy-16-methyl-3-oxoandrosta-1,4-diene-17-carboxylic acid

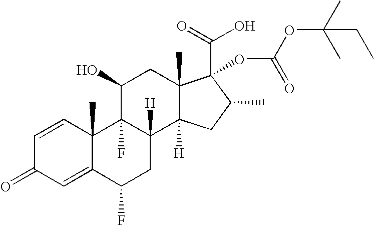

Prepared from bis(1,1-dimethylpropyl) dicarbonate using a method similar to that described for Intermediate 1. LCMS retention time 3.38 min.

### Intermediate 3: (6α,11β,16α,17α)-6,9-Difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylic acid

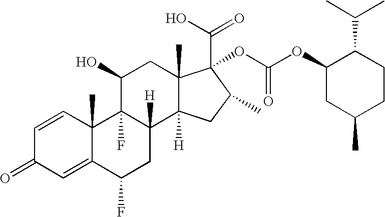

(1R)-(−)-Menthyl chloroformate (149 μl, 0.69 mmol) was added to a stirred solution of (6×,11β,16α,17α)-6,9-difluoro-11,17-dihydroxy-16-methyl-3-oxoandrosta-1,4-diene-17-carboxylic acid (250 mg, 0.63 mmol) in pyridine (5 ml) and the mixture was stirred at room temperature for 3.5 hours. The reaction was poured into 6M hydrochloric acid (30 ml) and the resulting precipitate was collected by filtration, washed with water (2×15 ml) and dried in vacuo at 40° C. to give the title compound (385 mg): LCMS retention time 3.92 min.

### Intermediate 4: (6α,11β,16α,17α)-17-({[(1-Ethylpropyl)oxy]carbonyl}oxy)-6,9-difluoro-11-hydroxy-16-methyl-3-oxoandrosta-1,4-diene-17-carboxylic acid

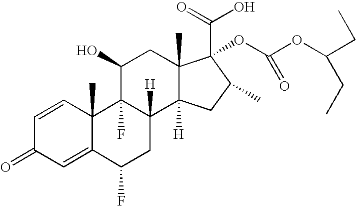

A solution of 3-pentanol (108 μl, 1 mmol) and pyridine (81 μl, 1 mmol) in anhydrous dichloromethane (2 ml) was added portionwise over 10 min to a stirred and cooled (ice) solution of triphosgene (98 mg, 0.33 mmol) in anhydrous dichloromethane (4 ml) under nitrogen. After 1 h, approximately half of the resulting chloroformate solution was added to a solution of (6α,11β,16α,17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature overnight. The solvent was evaporated in vacuo and the remaining residue stirred with 2M hydrochloric acid. The resulting precipitate was collected by filtration and dried in vacuo to give the title compound as a white solid (246 mg): LCMS retention time 3.42 min.

### Intermediate 5: (6α,11β,16α,17α)-6,9-Difluoro-11-hydroxy-16-methyl-17-[({[2-methyl-1-(1-methylethyl)propyl]oxy}carbonyl)oxy]-3-oxoandrosta-1,4-diene-17-carboxylic acid

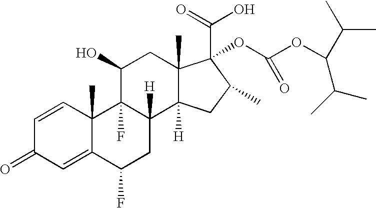

Prepared from 2,4-dimethyl-3-pentanol using a method similar to that described for Intermediate 4. LCMS retention time 3.58 min.

### Intermediate 6: (6α,11β,16α,17α)-17-({[(2-Ethylbutyl)oxy]carbonyl}oxy)-6,9-difluoro-11-hydroxy-16-methyl-3-oxoandrosta-1,4-diene-17-carboxylic acid

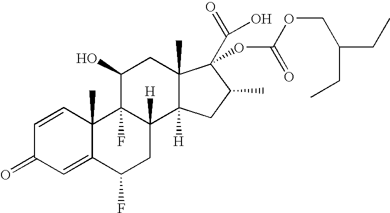

Prepared from 2-ethyl-1-butanol using a method similar to that described for Intermediate 4. LCMS retention time 3.63 min.

### Intermediate 7: (6α,11β,16α,17α)-17-({[(2,2-Dimethylpropyl)oxy]carbonyl}oxy)-6,9-difluoro-11-hydroxy-16-methyl-3-oxoandrosta-1,4-diene-17-carboxylic acid

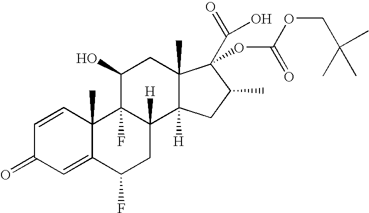

Prepared from 2,2-dimethyl-1-propanol using a method similar to that described for Intermediate 4. LCMS retention time 3.47 min.

### Intermediate 8: (6α,11β,16α,14α)-17-({[(1-Ethyl-2-methylpropyl)oxy]carbonyl}oxy)-6,9-difluoro-11-hydroxy-16-methyl-3-oxoandrosta-1,4-diene-17-carboxylic acid

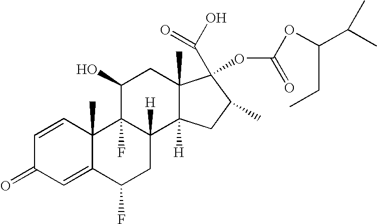

Prepared from 2-methyl-3-pentanol using a method similar to that described for Intermediate 4. LCMS retention time 3.54 min.

### Intermediate 9: (6α,11β,16α,17α)-17-({[(1,2-Dimethylpropyl)oxy]carbonyl}oxy)-6,9-difluoro-11-hydroxy-16-methyl-3-oxoandrosta-1,4-diene-17-carboxylic acid

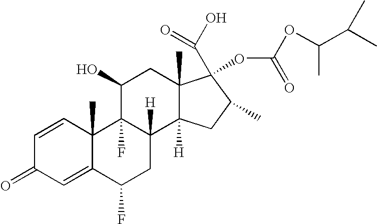

Prepared from 3-methyl-2-butanol using a method similar to that described for Intermediate 4. LCMS retention time 3.43 min.

### Intermediate 10: (6α,11β,16α,17α)-6,9-Difluoro-11-hydroxy-16-methyl-17-[({[(1SR,2RS)-2-methylcyclohexyl]oxy}carbonyl)oxy]-3-oxoandrosta-1,4-diene-17-carboxylic acid

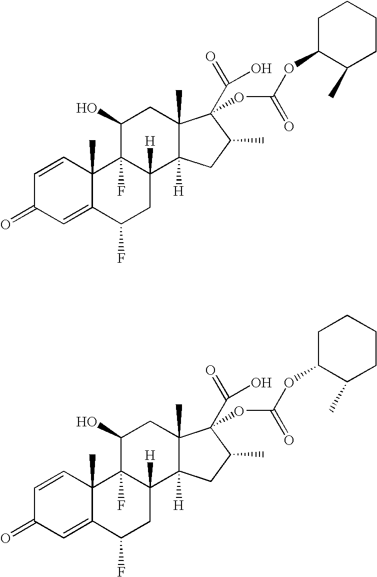

Prepared from racemic cis-2-methylcyclohexanol using a method similar to that described for Intermediate 4. LCMS retention time 3.59 min.

### Intermediate 11: (6α,11β,16α,17α)-6,9-Difluoro-11-hydroxy-16-methyl-17-[({[4-(1-methylethyl)cyclohexyl]oxy}carbonyl)oxy]-3-oxoandrosta-1,4-diene-17-carboxylic acid

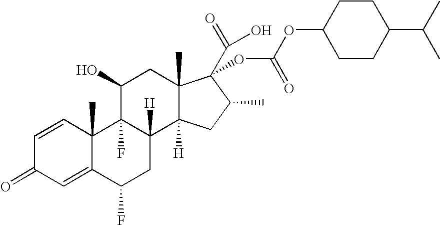

Prepared from cis/trans-4-(1-methylethyl)cyclohexanol using a method similar to that described for Intermediate 4. LCMS retention time 3.87 min.

### Intermediate 12: (6α,11β,16α,17α)-17-({[(1RS,2RS,4SR)-Bicyclo[2.2.1]hept-2-yloxy]carbonyl}oxy)-6,9-difluoro-11-hydroxy-16-methyl-3-oxoandrosta-1,4-diene-17-carboxylic acid

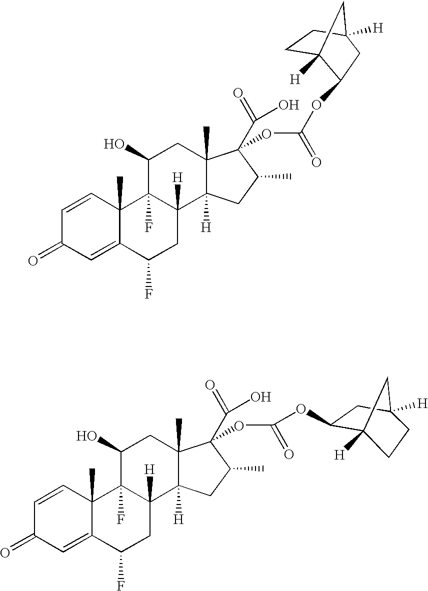

A solution of racemic exo-2-norborneol (113 mg, 1 mmol) and pyridine (81 μl, 1 mmol) in anhydrous dichloromethane (2 ml) was added portionwise over 10 min to a stirred and cooled (ice) solution of triphosgene (98 mg, 0.33 mmol) in anhydrous dichloromethane (4 ml) under nitrogen. After 1 h, approximately half of the resulting chloroformate solution was added to a solution of (6α,11β,16α, 17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature overnight. The remainder of the chloroformate solution was then added and after 2 hours the solvent was evaporated in vacuo and the remaining residue stirred with 2M hydrochloric acid. The resulting precipitate was collected by filtration and dried in vacuo to give the title compound as a white solid (254 mg): LCMS retention time 3.54 min.

### Intermediate 13: (6α,11β,16α,17α)-17-({[(1RS,2SR,4SR)-Bicyclo[2.2.1]hept-2-yloxy]carbonyl}oxy)-6,9-difluoro-11-hydroxy-16-methyl-3-oxoandrosta-1,4-diene-17-carboxylic acid

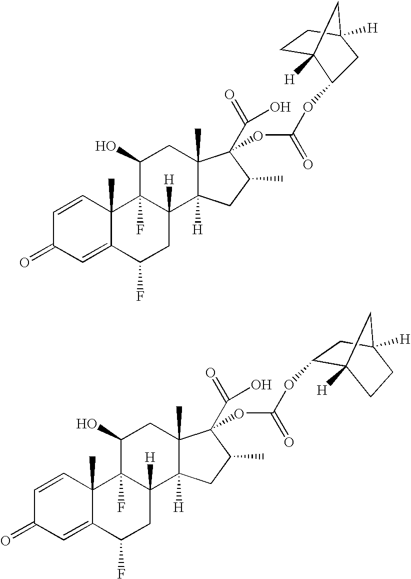

Prepared from racemic endo-2-norborneol using a method similar to that described for Intermediate 4. LCMS retention time 3.54 min.

### Intermediate 14: (6α,11β,16α,17α)-17-{[(Cycloheptyloxy)carbonyl]oxy}-6,9-difluoro-11-hydroxy-16-methyl-3-oxoandrosta-1,4-diene-17-carboxylic acid

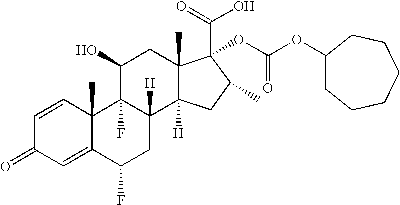

A solution of cycloheptanol (152 μl, 1.26 mmol) and pyridine (102 μl, 1.26 mmol) in anhydrous dichloromethane (2.5 ml) was added portionwise over 10 min to a stirred and cooled (ice) solution of triphosgene (125 mg, 0.42 mmol) in anhydrous dichloromethane (6 ml) under nitrogen. The ice bath was removed and after 1 h approximately half of the resulting chloroformate solution was added to a solution of (6α,11β,16α,17α)-6,9-difluoro-11,17-dihydroxy-16-methyl-3-oxoandrosta-1,4-diene-17-carboxylic acid (250 mg, 0.63 mmol) in pyridine (2 ml) and the mixture stirred at room temperature for approximately 3 hours. The remainder of the chloroformate solution was then added and after overnight stirring the reaction was partitioned between 5M hydrochloric acid and ethyl acetate. The organic layer was separated, washed with 1:1 brine:water and evaporated in vacuo to give the title compound as a white solid (341 mg): LCMS retention time 3.61 min.

### Intermediate 15: (6α,11β,16α,17α)-17-({[(Cyclopentylmethyl)oxy]-carbonyl}oxy)-6,9-difluoro-11-hydroxy-16-methyl-3-oxoandrosta-1,4-diene-17-carboxylic acid

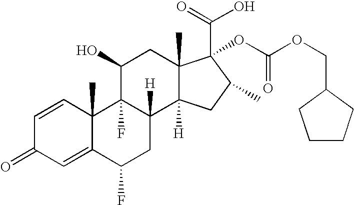

A solution of cyclopentanemethanol (108 μl, 1 mmol) and pyridine (81 μl, 1 mmol) in anhydrous dichloromethane (2 ml) was added portionwise over 10 min to a stirred and cooled (ice) solution of triphosgene (98 mg, 0.33 mmol) in anhydrous dichloromethane (4 ml) under nitrogen. After 1 h, approximately half of the resulting chloroformate solution was added to a solution of (6α,11β,16α,17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature for 3 hours. The remainder of the chloroformate solution was then added and after overnight stirring the solvent was evaporated in vacuo and the remaining residue stirred with 2M hydrochloric acid. The resulting precipitate was collected by filtration and dried in vacuo to give the title compound as a white solid (205 mg): LCMS retention time 3.52 min.

### Intermediate 16: (6α,11β,16α,17α)-17-{[(Cyclooctyloxy)carbonyl]oxy}-6,9-difluoro-11-hydroxy-16-methyl-3-oxoandrosta-1,4-diene-17-carboxylic acid

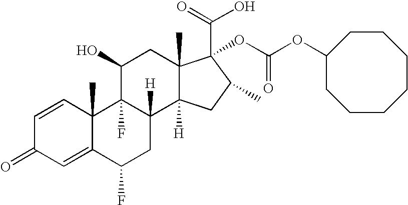

Prepared from cyclooctanol using a method similar to that described for Intermediate 15. LCMS retention time 3.71 min.

### Intermediate 17: (6α,11β,16α,17α)-17-[({[(1 S,3R,5S)-3,5-Dimethylcyclohexyl]oxy}carbonyl)oxy]-6,9-difluoro-11-hydroxy-16-methyl-3-oxoandrosta-1,4-diene-17-carboxylic acid

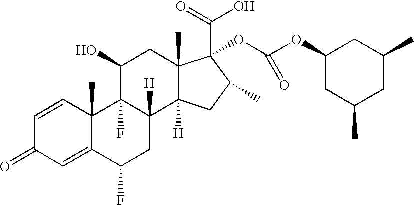

A solution of cis,cis,cis-3,5-dimethylcyclohexanol (144 μl, 1 mmol) and pyridine (81 μl, 1 mmol) in anhydrous dichloromethane (2 ml) was added portionwise over 10 min to a stirred and cooled (ice) solution of triphosgene (98 mg, 0.33 mmol) in anhydrous dichloromethane (4 ml) under nitrogen. After 1 h, approximately half of the resulting chloroformate solution was added to a solution of (6α,11β,16α,17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature overnight. The remainder of the chloroformate solution was then added, followed by addition of a further two equivalents (1 mmol) of freshly prepared chloroformate solution. After 3 hours the solvent was evaporated in vacuo and the remaining residue stirred with 2M hydrochloric acid. The resulting precipitate was collected by filtration and dried in vacuo to give the title compound as a white solid (313 mg): LCMS retention time 3.76 min.

### Intermediate 18: (6α,11β,16α,17α)-6,9-Difluoro-11-hydroxy-16-methyl-17-[({[(1 RS,2RS)-2-(methyloxy)cyclohexyl]oxy}carbonyl)oxy]-3-oxoandrosta-1,4-diene-17-carboxylic acid

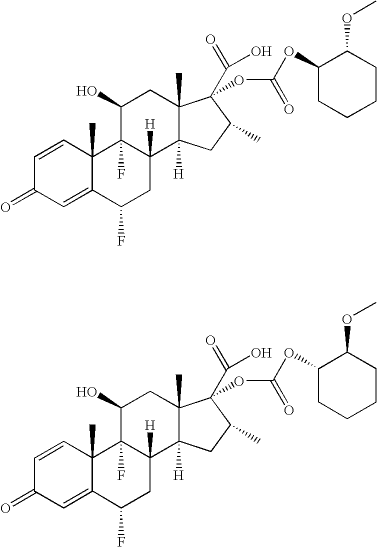

Prepared from racemic trans-2-methoxy-cyclohexanol (G. H. Posner et al., (1975) Tetrahedron Letters, 16, Issue 42, 3589-3600) using a method similar to that described for Intermediate 12. LCMS retention time 3.28 min. and 3.36 min.

### Intermediate 19: (6α,11β,16α,17α)-17-({[(3,3-Dimethylcyclohexyl)oxy]carbonyl}oxy)-6,9-difluoro-11-hydroxy-16-methyl-3-oxoandrosta-1,4-diene-17-carboxylic acid

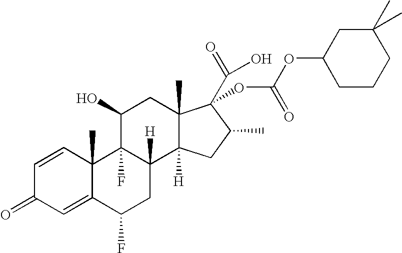

Prepared from 3,3-dimethylcyclohexanol using a method similar to that described for Intermediate 4. LCMS retention time 3.71 min.

### Intermediate 20: (6α,11β,16α,17α)-17-({[(1-Cyclopentylpropyl)oxy]carbonyl}oxy)-6,9-difluoro-11-hydroxy-16-methyl-3-oxoandrosta-1,4-diene-17-carboxylic acid

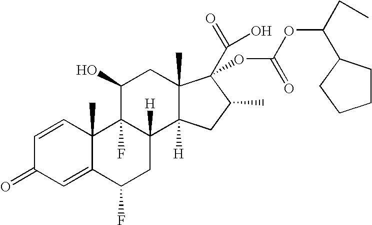

Prepared from 1-cyclopentyl-1-propanol using a method similar to that described for Intermediate 4. LCMS retention time 3.71 min. and 3.73 min.

### Intermediate 21: (6α,11β,16α,17α)-17-({[(1-Cyclopentylethyl)oxy]carbonyl}oxy)-6,9-difluoro-11-hydroxy-16-methyl-3-oxoandrosta-1,4-diene-17-carboxylic acid

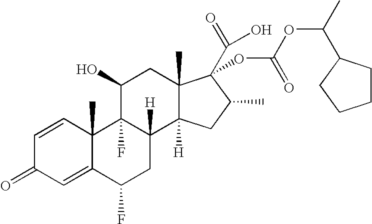

Prepared from 1-cyclopentylethanol using a method similar to that described for Intermediate 4. LCMS retention time 3.61 min. and 3.64 min.

### Intermediate 22: (6α,11β,16α,17α)-6,9-Difluoro-11-hydroxy-16-methyl-3-oxo-17-({[(1-propylbutyl)oxy]-carbonyl}oxy)androsta-1,4-diene-17-carboxylic acid

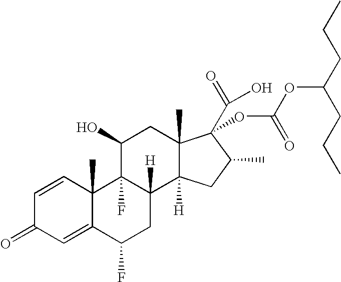

Prepared from 4-heptanol using a method similar to that described for Intermediate 4. LCMS retention time 3.65 min.

### Intermediate 23: (6α,11β,16α,17α)-6,9-Difluoro-11-hydroxy-16-methyl-3-oxo-17-({[(1,2,2-trimethylpropyl)oxy]carbonyl}oxy)androsta-1,4-diene-17-carboxylic acid

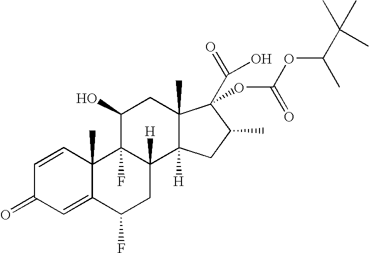

Prepared from 3,3-dimethyl-2-butanol using a method similar to that described for Intermediate 4. LCMS retention time 3.44 min. and 3.54 min.

### Intermediate 24: (6α,11β,16α,17α)-6,9-Difluoro-11-hydroxy-16-methyl-3-oxo-17-[({[(2,2,3,3-tetramethylcyclopropyl)methyl]oxy}carbonyl)oxy]androsta-1,4-diene-17-carboxylic acid

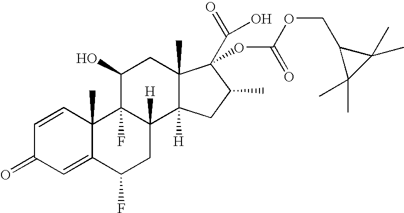

A solution of (2,2,3,3-tetramethylcyclopropyl)methanol (P. S. Wharton et al., (1965) Journal of Organic Chemistry, 30, 1681-1684) (128 mg, 1 mmol) and pyridine (81 μl, 1 mmol) in anhydrous dichloromethane (2 ml) was added to a stirred and cooled (ice) solution of triphosgene (98 mg, 0.33 mmol) in anhydrous dichloromethane (4 ml) under nitrogen. After 1 h, approximately half of the resulting chloroformate solution was added to a solution of (6α,11β,16α,17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature for 3 hours. The remainder of the chloroformate solution was then added and after overnight stirring a further two equivalents (1 mmol) of freshly prepared chloroformate solution was added. After stirring for 72 hours the solvent was evaporated in vacuo and the remaining residue stirred with 2M hydrochloric acid. The resulting precipitate was collected by filtration to give the title compound as a white solid (180 mg): LCMS retention time 3.68 min.

### Intermediate 25: (6α,11β,16α,17α)-6,9-Difluoro-11-hydroxy-16-methyl-17-[({[1-(1-methylethyl)butyl]oxy}carbonyl)oxy]-3-oxoandrosta-1,4-diene-17-carboxylic acid

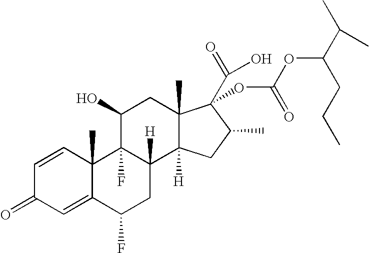

Prepared from 2-methyl-3-hexanol using a method similar to that described for Intermediate 4. LCMS retention time 3.66 min.

### Intermediate 26: (6α,11β,16α,17α)-6,9-Difluoro-11-hydroxy-16-methyl-3-oxo-17-({[(2,2,3,3-tetramethylcyclopropyl)oxy]carbonyl}oxy)androsta-1,4-diene-17-carboxylic acid

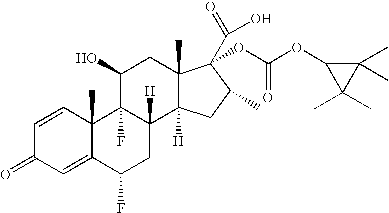

A solution of 2,2,3,3-tetramethylcyclopropanol (114 mg, 1 mmol) and pyridine (162 μl, 2 mmol) in anhydrous dichloromethane (2 ml) was added portionwise over 5 min to a stirred and cooled (ice) solution of triphosgene (105 mg, 0.35 mmol) in anhydrous dichloromethane (3 ml) under nitrogen. After 1 h the resulting chloroformate solution was added to an ice cooled solution of (6α,11β,16α,17α)-6,9-difluoro-11,17-dihydroxy-16-methyl-3-oxoandrosta-1,4-diene-17-carboxylic acid (200 mg, 0.5 mmol) in pyridine (2 ml) and the mixture stirred at room temperature overnight. The reaction was then evaporated in vacuo and partitioned between 2M hydrochloric acid and ethyl acetate. The organic layer was separated, filtered through a hydrophobic frit and evaporated in vacuo. The crude product was purified on a 5 g silica Bond Elut cartridge using a 0-100% ethyl acetate in cyclohexane gradient to give the title compound as a pale yellow foam (240 mg): LCMS retention time 3.65 min.

### Intermediate 27: (6α,11β,16α,17α)-6,9-Difluoro-11-hydroxy-16-methyl-3-oxo-17-[({[(1S,2R,4S)-1,7,7-trimethylbicyclo[2.2.1]hept-2-yl]oxy}carbonyl)oxy]androsta-1,4-diene-17-carboxylic acid

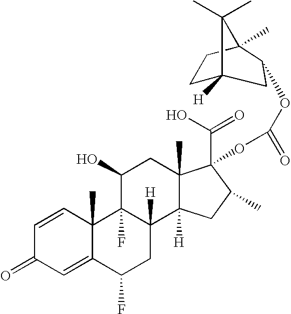

Prepared from (−) Borneol using a method similar to that described for Intermediate 4. LCMS retention time 3.87 min.

### Intermediate 28: (6α,11β,16α,17α)-6,9-Difluoro-1-hydroxy-16-methyl-3-oxo-17-[({[(1R,2S,4R)-1,7,7-trimethylbicyclo[2.2.1]hept-2-yl]oxy}carbonyl)oxy]androsta-1,4-diene-17-carboxylic acid

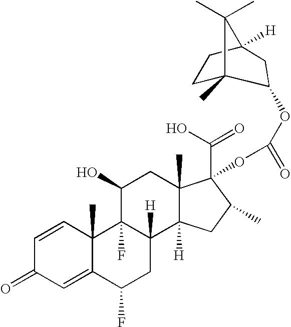

Prepared from (+) Borneol using a method similar to that described for Intermediate 4. LCMS retention time 3.81 min.

### Intermediate 29: (6×, 110.16a, 17a)-6,9-Difluoro-11-hydroxy-16-methyl-3-oxo-17-[({[(1RS,2RS,4RS)-1,7,7-trimethylbicyclo[2.2.1]hept-2-yl]oxy}carbonyl)oxy]androsta-1,4-diene-17-carboxylic acid

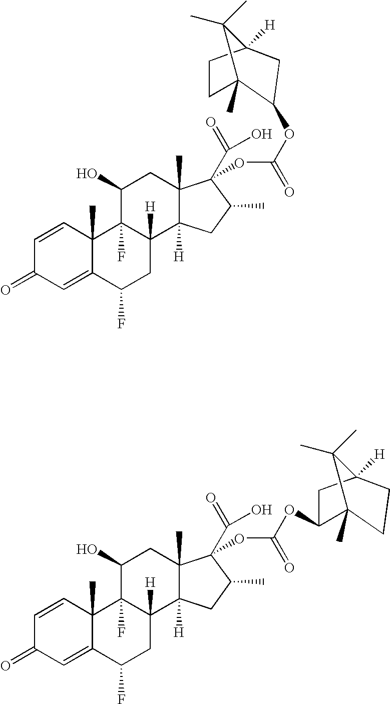

Prepared from (+/−) Isoborneol using a method similar to that described for Intermediate 12. LCMS retention time 3.85 min.

Intermediate 30: (6α,11β,16α,17α)-17-[({[(1R,2R,4S)-3,3-Dimethylbicyclo[2.2.1]hept-2-yl]oxy}carbonyl)oxy]-6,9-difluoro-11-hydroxy-16-methyl-3-oxoandrosta-1,4-diene-17-carboxylic acid

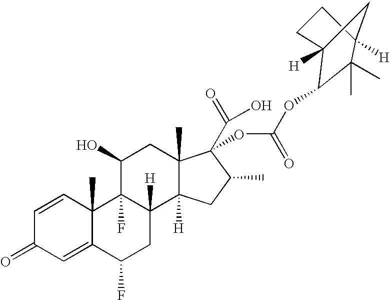

Prepared from (1R,2R,4S)-3,3-dimethylbicyclo[2.2.1]heptan-2-ol (P. Veeraraghavan Ramachandran et al., (1996) Journal of Organic Chemistry, 61, Issue 1, 95-99) using a method similar to that described for Intermediate 4. LCMS retention time 3.84 min.

### Intermediate 31: (6α,11β,16α,17α)-17-{[(Cyclopentyloxy)carbonyl]oxy}-6,9-difluoro-11-hydroxy-16-methyl-3-oxoandrosta-1,4-diene-17-carboxylic acid

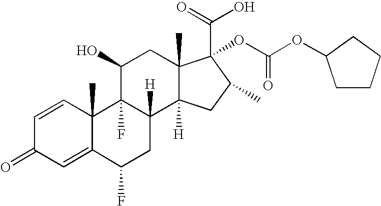

Cyclopentyl chloroformate (211 mg, 1.44 mmol) was added to a stirred solution of (6α,11β,16α,17α)-6,9-difluoro-11,17-dihydroxy-16-methyl-3-oxoandrosta-1,4-diene-17-carboxylic acid (500 mg, 1.26 mmol) in pyridine (5 ml) and the mixture was stirred at room temperature under nitrogen for 12 hours. The reaction was poured into 6M hydrochloric acid (40 ml) and the resulting precipitate extracted into ethyl acetate (2×40 ml). The organic phase was separated, washed with 2M hydrochloric acid (2×50 ml), dried over magnesium sulphate, filtered and evaporated in vacuo to give the title compound (720 mg): LCMS retention time 3.50 min.

### Intermediate 32: (6α,11β,16α,17α)-6,9-Difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylic acid

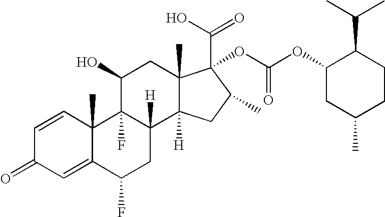

Prepared from (1S)-(+)-Menthyl chloroformate using a method similar to that described for Intermediate 3. LCMS retention time 3.89 min

### Intermediate 33: (6α,11β,16α,17α)-6,9-Difluoro-11-hydroxy-16-methyl-3-oxo-17-[({[(1R,2R,4S)-1,3,3-trimethylbicyclo[2.2.1]hept-2-yl]oxy}carbonyl)oxy]androsta-1,4-diene-17-carboxylic acid

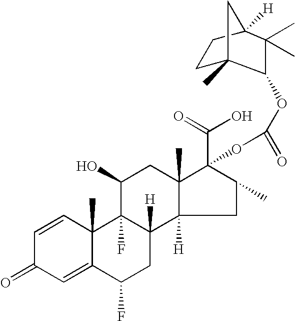

Prepared from (1R)-endo-(+)-fenchyl alcohol using a method similar to that described for Intermediate 4. LCMS retention time 3.87 min.

### Intermediate 34: (6α,11β,16α,17α)-6,9-Difluoro-11-hydroxy-16-methyl-3-oxo-17-[({[(1R,2R,3R,5S)-2,6,6-trimethylbicyclo[3.1.1]hept-3-yl]oxy}carbonyl)oxy]androsta-1,4-diene-17-carboxylic acid

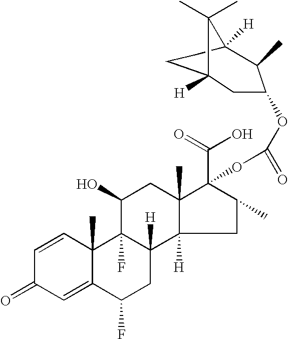

Prepared from (−)-isopinocampheol using a method similar to that described for Intermediate 4. LCMS retention time 3.87 min.

### Intermediate 35: (6α,11β,16α,17α)-6,9-Difluoro-11-hydroxy-16-methyl-3-oxo-17-[({[(1S,2S,3S,5R)-2,6,6-trimethylbicyclo[3.1.1]hept-3-yl]oxy}carbonyl)oxy]androsta-1,4-diene-17-carboxylic acid

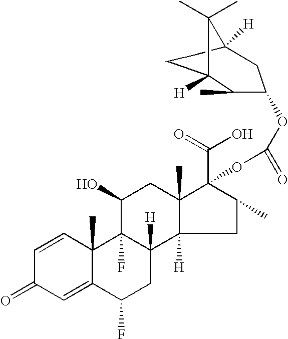

Prepared from (+)-isopinocampheol using a method similar to that described for Intermediate 4. LCMS retention time 3.86 min.

### Intermediate 36: (6α,11β,16α,17α)-17-({[(cis-4-Ethylcyclohexyl)oxy]carbonyl}oxy)-6,9-difluoro-11-hydroxy-16-methyl-3-oxoandrosta-1,4-diene-17-carboxylic acid

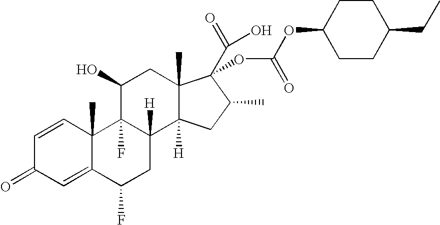

Prepared from cis-4-ethylcyclohexanol using a method similar to that described for Intermediate 4. LCMS retention time 3.76 min.

### Intermediate 37: (6α,11β,16α,17α)-17-({[(trans-4-Ethylcyclohexyl)oxy]carbonyl}oxy)-6,9-difluoro-11-hydroxy-16-methyl-3-oxoandrosta-1,4-diene-17-carboxylic acid

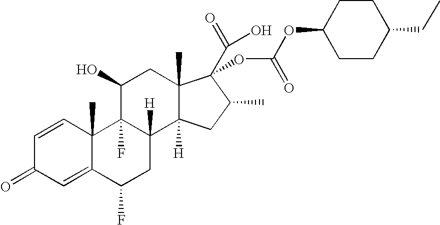

Prepared from trans-4-ethylcyclohexanol using a method similar to that described for Intermediate 4. LCMS retention time 3.78 min.

### Intermediate 38: (6α,11β,16α,17α)-17-({[(1-Ethyl-2,2-dimethylpropyl)oxy]carbonyl}oxy)-6,9-difluoro-11-hydroxy-16-methyl-3-oxoandrosta-1,4-diene-17-carboxylic acid

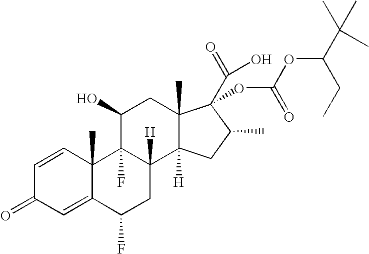

Prepared from 2,2-dimethyl-3-pentanol using a method similar to that described for Intermediate 4. LCMS retention time 3.61 min.

### EXAMPLES

### Example 1

### Fluoromethyl(6α,11β,16α,17α)-17-({[(1,1-dimethylethyl)oxy]carbony}oxy)-6,9-difluoro-11-hydroxy-16-methyl-3-oxoandrosta-1,4-diene-17-carboxylate

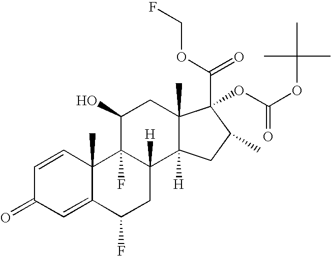

Sodium carbonate (668 mg, 6.3 mmol) was added to a solution of (6α,11β,16α,17α)-17-({[(1,1-dimethylethyl)oxy]carbonyl}oxy)-6,9-difluoro-11-hydroxy-16-methyl-3-oxoandrosta-1,4-diene-17-carboxylic acid (Intermediate 1) (250 mg, 0.5 mmol) in anhydrous N,N-dimethylformamide (5 ml) and the mixture cooled to −20° C. Bromofluoromethane (96 μl, 1.7 mmol) was added and the reaction stirred at −30° C. to −20° C. for 2 hours before being allowed to warm to room temperature overnight. The reaction was then treated with diethylamine (500 μL, 7.56 mmol) and added dropwise to 6M hydrochloric acid (30 ml). The resulting precipitate was collected by filtration, washed with 2M hydrochloric acid (10 ml) followed by water (3×10 ml) and dried in vacuo at 50° C. The crude product was purified on a 10 g silica Bond Elut cartridge using a 11-50% ethyl acetate in cyclohexane gradient to give the title compound (174 mg): LCMS retention time 3.55 min, m/z 529 MH⁺

### Example 2

### Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate

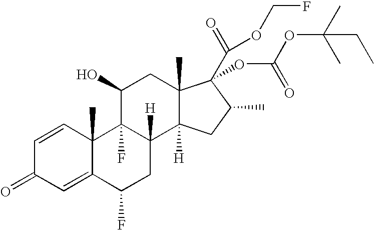

Sodium carbonate (208 mg, 1.96 mmol) was added to a stirred solution of (6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylic acid (Intermediate 2) (100 mg, 0.2 mmol) in anhydrous N,N-dimethylformamide (3 ml) and after stirring at room temperature for 15 minutes the mixture was cooled to −30° C. under nitrogen. Bromofluoromethane (30 μl, 0.53 mmol) was added and the reaction allowed to warm to room temperature overnight. The reaction was then treated with diethylamine (26 μl, 0.34 mmol) and added dropwise to 2M hydrochloric acid (20 ml). The resulting precipitate was extracted into ethyl acetate which was dried over anhydrous magnesium sulphate, filtered and evaporated in vacuo. The crude product was purified on a 5 g silica Bond Elut cartridge using a 0-100% ethyl acetate in cyclohexane gradient over 40 minutes to give the title compound (146 mg): LCMS retention time 3.67 min, m/z 543 MH⁺

### Example 3

### Fluoromethyl(6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-14-diene-17-carboxylate

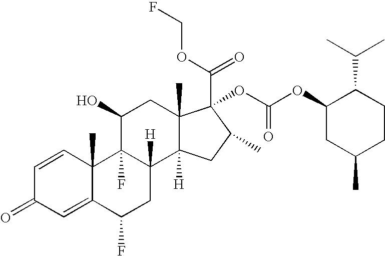

Example 3 was prepared from (6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylic acid (Intermediate 3) using a method similar to that described for Example 1. LCMS retention time 4.07 min, m/z 611 MH⁺

### Example 4

### Fluoromethyl(6α,11β,16α,17α)-17-({[(1-ethylpropyl)oxy]carbonyl}oxy)-6,9-difluoro-11-hydroxy-16-methyl-3-oxoandrosta-1,4-diene-17-carboxylate

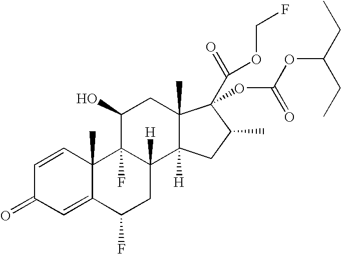

Sodium carbonate (311 mg, 2.93 mmol) was added to a stirred solution of (6α,11β,16α,17α)-17-({[(1-ethylpropyl)oxy]carbonyl}oxy-6,9-difluoro-11-hydroxy-16-methyl-3-oxoandrosta-1,4-diene-17-carboxylic acid (Intermediate 4) (150 mg, 0.29 mmol) in anhydrous N,N-dimethylformamide (3 ml) and after stirring at room temperature for 15 minutes the mixture was cooled to −30° C. under nitrogen. Bromofluoromethane (45 μl, 0.79 mmol) was added and the reaction stirred at −25 to −35° C. for 2 hours. Further bromofluoromethane (45 μl, 0.79 mmol) was added and the reaction allowed to warm to room temperature overnight. The reaction was then treated with diethylamine (87 μl, 1.29 mmol) and added dropwise to 2M hydrochloric acid. The resulting precipitate was extracted into ethyl acetate which was dried over anhydrous magnesium sulphate, filtered and evaporated in vacuo. The crude product was purified on a 5 g silica Bond Elut cartridge eluted using 1:1 diethylether:cyclohexane to give the title compound (111 mg): LCMS retention time 3.60 min, m/z 543 MH⁺

### Example 5

### Fluoromethyl(6α,11β,16α,17α)-6,9-difluoro-11-hydroxy-16-methyl-17-[({[2-methyl-1-(1-methylethyl)propyl]oxy}carbonyl)oxy]-3-oxoandrosta-1,4-diene-17-carboxylate

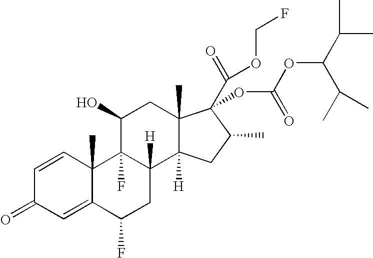

Example 5 was prepared from (6α,11β,16α,17α)-6,9-difluoro-11-hydroxy-16-methyl-17-[({[2-methyl-1-(1-methylethyl)propyl]oxy}carbonyl)oxy]-3-oxoandrosta-1,4-diene-17-carboxylic acid (Intermediate 5) using a method similar to that described for Example 4. The crude product was purified on a 5 g silica Bond Elut cartridge using 1:1 diethylether:cyclohexane to give the title compound: LCMS retention time 3.76 min, m/z 571 MH⁺

### Example 6

### Fluoromethyl(6α,11β,16α,17α)-17-({[(2-ethylbutyl)oxy]carbonyl}oxy)-6,9-difluoro-11-hydroxy-16-methyl-3-oxoandrosta-1,4-diene-17-carboxylate

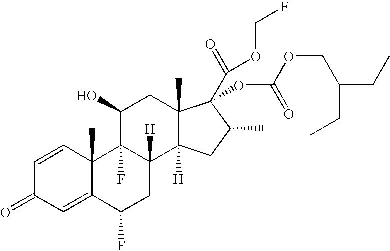

Sodium carbonate (304 mg, 2.86 mmol) was added to a stirred solution of (6α,11β,16α,17α)-17-({[(2-ethylbutyl)oxy]carbonyl}oxy)-6,9-difluoro-11-hydroxy-16-methyl-3-oxoandrosta-1,4-diene-17-carboxylic acid (Intermediate 6) (150 mg, 0.29 mmol) in anhydrous N,N-dimethylformamide (3 ml) and after stirring at room temperature for 15 minutes the mixture was cooled to −30° C. under nitrogen. Bromofluoromethane (44 μl, 0.77 mmol) was added and the reaction stirred at −25 to −35° C. for 2 hours followed by overnight at room temperature. Further bromofluoromethane (22 μl, 0.39 mmol) was then added and the reaction stirred at room temperature for 2 hours. Again, further bromofluoromethane (22 μl, 0.39 mmol) was added and the reaction stirred at room temperature overnight. The reaction was then treated with diethylamine (85 μl, 1.26 mmol) and added dropwise to 2M hydrochloric acid (20 ml). The resulting precipitate was extracted into ethyl acetate which was dried over anhydrous magnesium sulphate, filtered and evaporated in vacuo. The crude product was purified on a 10 g silica Bond Elut cartridge eluted using a 0-100% diethylether in cyclohexane gradient over 40 minutes to give the title compound (70 mg): LCMS retention time 3.78 min, m/z 557 MH⁺

### Example 7

### Fluoromethyl(6α,11β,16α,17α)-17-({[(2,2-dimethylpropyl)oxy]carbonyl}oxy)-6,9-difluoro-11-hydroxy-16-methyl-3-oxoandrosta-1,4-diene-17-carboxylate

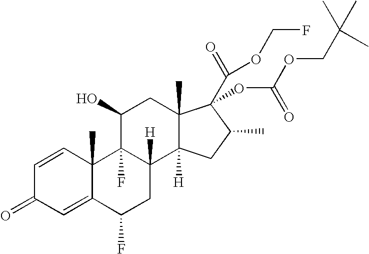

Example 7 was prepared from (6α,11β,16α,17α)-17-({[(2,2-dimethylpropyl)oxy]carbonyl}oxy)-6,9-difluoro-11-hydroxy-16-methyl-3-oxoandrosta-1,4-diene-17-carboxylic acid (Intermediate 7) using a method similar to that described for Example 4. The crude product was purified on a 10 g silica Bond Elut cartridge eluted using 0-100% diethylether in cyclohexane gradient over 40 minutes to give the title compound: LCMS retention time 3.61 min, m/z 543 MH⁺

### Example 8

### Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate

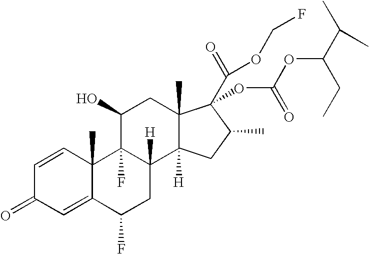

Sodium carbonate (304 mg, 2.86 mmol) was added to a stirred solution of (6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylic acid (Intermediate 8) (150 mg, 0.29 mmol) in anhydrous N,N-dimethylformamide (3 ml) and after stirring at room temperature for 15 minutes the mixture was cooled to −30° C. under nitrogen. Bromofluoromethane (44 μl, 0.77 mmol) was added and the reaction stirred at −25 to −35° C. for 2 hours followed by overnight at room temperature. Further bromofluoromethane (22 μl, 0.39 mmol) was then added and the reaction stirred at room temperature overnight. The reaction was then treated with diethylamine (85 μl, 1.26 mmol) and added dropwise to 2M hydrochloric acid (20 ml). The resulting precipitate was extracted into ethyl acetate which was dried over anhydrous magnesium sulphate, filtered and evaporated in vacuo. The crude product was purified on a 10 g silica Bond Elut cartridge eluted using a 0-100% diethylether in cyclohexane gradient over 40 minutes to give the title compound as a mixture of diastereomers (86 mg): LCMS retention time 3.67 min, m/z 557 MH⁺

### Example 9

### Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate

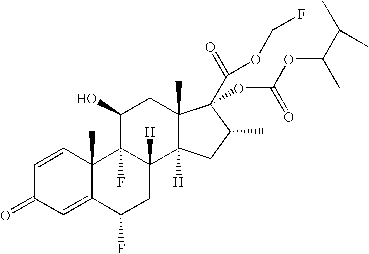

Example 9 was prepared as a mixture of diastereomers from (6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylic acid (Intermediate 9) using a method similar to that described for Example 2. The crude product was purified on a 10 g silica Bond Elut cartridge eluted using a 0-100% diethylether in cyclohexane gradient over 40 minutes to give the title compound: LCMS retention time 3.58 min, m/z 543 MH⁺

### Example 10

### Fluoromethyl(6α,11β,16α,17α)-6,9-difluoro-11-hydroxy-16-methyl-17-[({[(1SR,2RS)-2-methylcyclohexyl]oxy}carbonyl)oxy]-3-oxoandrosta-1,4-diene-17-carboxylate

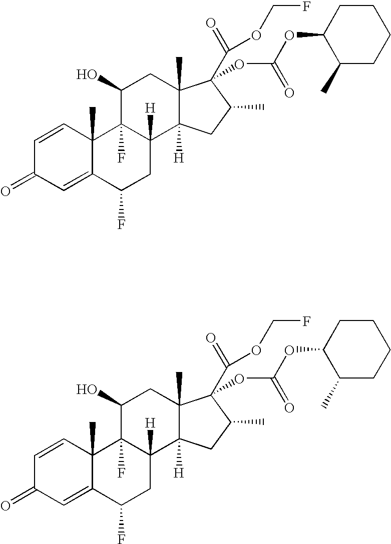

Sodium carbonate (297 mg, 2.8 mmol) was added to a stirred solution of (6α,11β,16α,17α)-6,9-difluoro-11-hydroxy-16-methyl-17-[({[(1SR,2RS)-2-methylcyclohexyl]oxy}carbonyl)oxy]-3-oxoandrosta-1,4-diene-17-carboxylic acid (Intermediate 10) (150 mg, 0.28 mmol) in anhydrous N,N-dimethylformamide (3 ml) and after stirring at room temperature for 15 minutes the mixture was cooled to −30° C. under nitrogen. Bromofluoromethane (43 μl, 0.76 mmol) was added and the reaction stirred at −25 to −35° C. for 2 hours. Further bromofluoromethane (43 μl, 0.76 mmol) was then added and the reaction stirred at room temperature overnight. The reaction was then treated with diethylamine (82 μl, 1.23 mmol) and added dropwise to 2M hydrochloric acid. The resulting precipitate was filtered and dried in vacuo to give the title compound as a mixture of diastereomers (94 mg).

The diastereomers were then separated using normal phase HPLC to give:

### Example 10A

### LCMS retention time 3.80 min, m/z 569 MH⁺. ¹H-NMR: (DMSO-d₆, 400 MHz) 17β fluoromethylene protons δ 5.85 (dd, 50.5, 2 Hz) and δ 5.70 (dd, 50.5, 2 Hz)

### Example 10B

### LCMS retention time 3.80 min, m/z 569 MH⁺. ¹H-NMR: (DMSO-d₆, 400 MHz) 17β fluoromethylene protons δ 5.85 (dd, 50.5, 2 Hz) and δ 5.74 (dd, 50.5, 2 Hz)

### Example 11

### Fluoromethyl(6α,11β,16α,17α)-6,9-difluoro-11-hydroxy-16-methyl-17-[({[4-(1-methylethyl)cyclohexyl]oxy}carbonyl)oxy]-3-oxoandrosta-1,4-diene-17-carboxylate

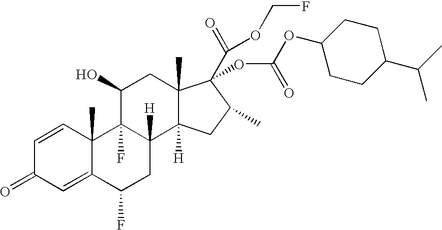

Sodium carbonate (188 mg, 1.77 mmol) was added to a stirred solution of (6α,11β,16α,17α)-6,9-difluoro-11-hydroxy-16-methyl-17-[({[4-(1-methylethyl)cyclohexyl]oxy}carbonyl)oxy]-3-oxoandrosta-1,4-diene-17-carboxylic acid (Intermediate 11) (100 mg, 0.18 mmol) in anhydrous N,N-dimethylformamide (3 ml) and after stirring at room temperature for 15 minutes the mixture was cooled to −30° C. under nitrogen. Bromofluoromethane (27 μl, 0.48 mmol) was added and the reaction stirred at −25 to −35° C. for 2.5 hours followed by overnight at room temperature. The reaction was then treated with diethylamine (20 μl, 0.3 mmol) and added dropwise to 2M hydrochloric acid. The resulting precipitate was filtered and dried in vacuo to give the title compound as a ca 4:1 mixture of diastereomers (39 mg).

The diastereomers were then separated by reverse phase HPLC—isocratic conditions—eluting with 55% mobile phase B, run time 45 min, flow rate 20 ml/min.

Mobile phase A—water/0.1% formic acid v/v

Mobile phase B—95% aq Acetonitrile/0.05% formic acid v/v

### Example 11A

### (Minor Isomer) LCMS Retention Time 3.90 min, m/z 597 MH⁺

### Example 11B

### (Major Isomer) LCMS Retention Time 3.97 min, m/z 597 MH⁺

### Example 12

### Fluoromethyl(6α,11β,16α,17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylate

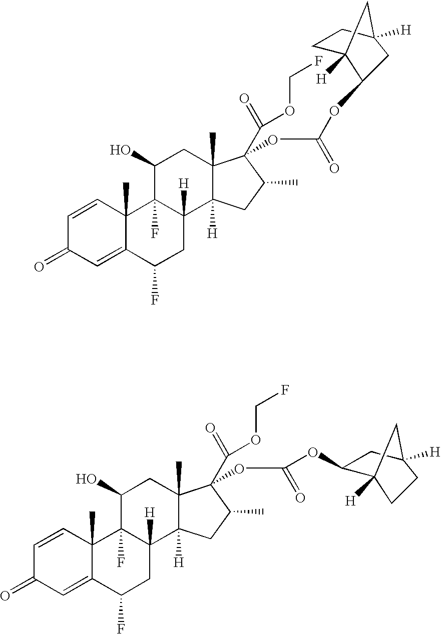

Example 12 was prepared as a mixture of diastereomers from (6α,11β,16α17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylic acid (Intermediate 12) using a method similar to that described for Example 10.

The diastereomers were then separated using a 2×25 cm Chiralpak AD column eluting with 10% isopropyl alcohol in heptane with a flow rate of 20 ml/min.

### Example 12A

### On analytical chiral HPLC (25×0.46 cm Chiralpak AD column, 10% isopropyl alcohol in heptane with a flow rate of 1 ml/min) showed a retention time 17.2 min. LCMS retention time 3.74 min, m/z 567 MH⁺

### Example 12B

### On analytical chiral HPLC (25×0.46 cm Chiralpak AD column, 10% isopropyl alcohol in heptane with a flow rate of 1 ml/min) showed a retention time 21.8 min. LCMS retention time 3.73 min, m/z 567 MH⁺

### Example 13

### Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate

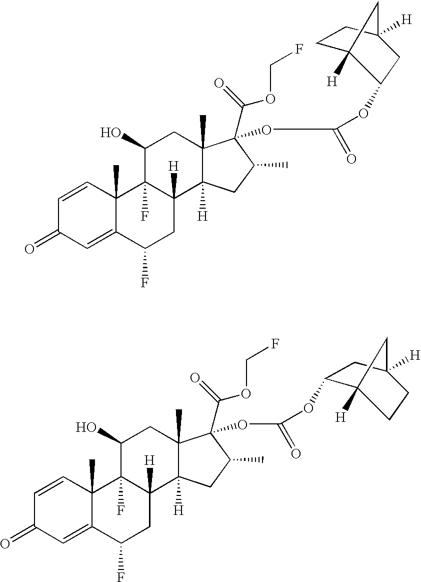

Example 13 was prepared as a mixture of diastereomers from (6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylic acid (Intermediate 13) using a method similar to that described for Example 10. LCMS retention time 3.77 min, m/z 567 MH⁺

### Example 14

### Fluoromethyl(6α,11β,16α,17α)-17-{[(cycloheptyloxy)carbonyl]oxy}-6,9-difluoro-11-hydroxy-16-methyl-3-oxoandrosta-1,4-diene-17-carboxylate

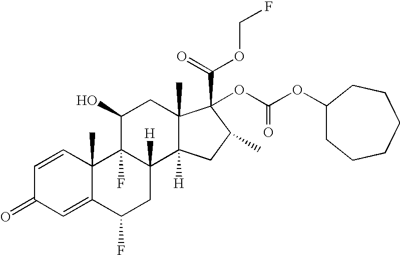

Sodium carbonate (680 mg, 6.4 mmol) was added to a stirred solution of (6α,11β,16α,17α)-17-{[(cycloheptyloxy)carbonyl]oxy}-6,9-difluoro-11-hydroxy-16-methyl-3-oxoandrosta-1,4-diene-17-carboxylic acid (Intermediate 14) (341 mg, 0.64 mmol) in anhydrous N,N-dimethylformamide (5 ml) and after stirring at room temperature for 15 minutes the mixture was cooled to −30° C. under nitrogen. Bromofluoromethane (98 μl, 1.73 mmol) was added and the reaction stirred at −25 to −35° C. for 1 hour. Further bromofluoromethane (98 μl, 1.73 mmol) was then added and the reaction stirred at −25 to −35° C. for 5 hours. Again, further bromofluoromethane (98 μl, 1.73 mmol) was added to the reaction mixture which was stirred at room temperature for 72 hours. The reaction was then treated with diethylamine (470 μl, 7.1 mmol) and added dropwise to 5M hydrochloric acid. The resulting precipitate was filtered and dried in vacuo to give the title compound (287 mg): LCMS retention time 3.79 min, m/z 569 MH⁺

### Example 15

### Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate

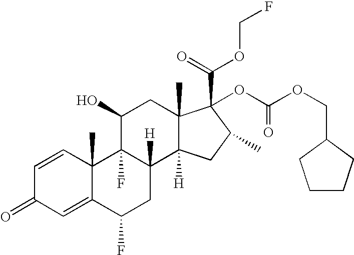

Example 15 was prepared from (6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylic acid (Intermediate 15) using a method similar to that described for Example 10. The crude product was purified on a 2 g silica Bond Elut cartridge eluted using a 0-100% diethylether in cyclohexane gradient over 9 minutes to give the title compound: LCMS retention time 3.71 min, m/z 555 MH⁺

### Example 16

### Fluoromethyl(6α,11β,16α,17α)-17-{[(cyclooctyloxy)carbonyl]oxy}-6,9-difluoro-11-hydroxy-16-methyl-3-oxoandrosta-1,4-diene-17-carboxylate

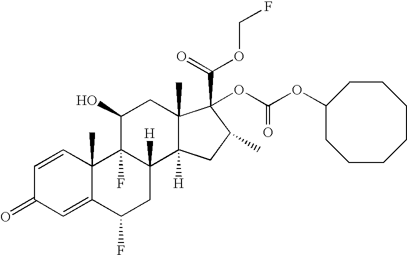

Example 16 was prepared from (6α,11β,16α,17α)-17-{[(cyclooctyloxy)carbonyl]oxy}-6,9-difluoro-11-hydroxy-16-methyl-3-oxoandrosta-1,4-diene-17-carboxylic acid (Intermediate 16) using a method similar to that described for Example 10. The crude product was purified on a 2 g silica Bond Elut cartridge eluted using a 0-100% diethylether in cyclohexane gradient over 9 minutes to give the title compound: LCMS retention time 3.87 min, m/z 583 MH⁺

### Example 17

### Fluoromethyl(6α,11β,16α,17α)-17-[({[(1S,3R,5S)-3,5-dimethylcyclohexyl]oxy}carbonyl)oxy]-6,9-difluoro-11-hydroxy-16-methyl-3-oxoandrosta-1,4-diene-17-carboxylate

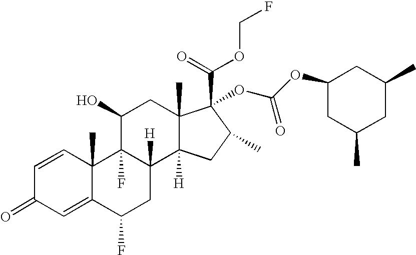

Example 17 was prepared from (6α,11β,16α,17α)-17-[({[(1S,3R,5S)-3,5-Dimethylcyclohexyl]oxy}carbonyl)oxy]-6,9-difluoro-11-hydroxy-16-methyl-3-oxoandrosta-1,4-diene-17-carboxylic acid (Intermediate 17) using a method similar to that described for Example 11. The crude product was purified on a silica biotage cartridge eluted using 25% ethyl acetate in cyclohexane to give the title compound: LCMS retention time 3.90 min, m/z 583 MH⁺

### Example 18

### Fluoromethyl(6α,11β,16α,17α)-6,9-difluoro-11-hydroxy-16-methyl-17-[({[(1RS,2RS)-2-(methyloxy)cyclohexyl]oxy}carbonyl)oxy]-3-oxoandrosta-1,4-diene-17-carboxylate

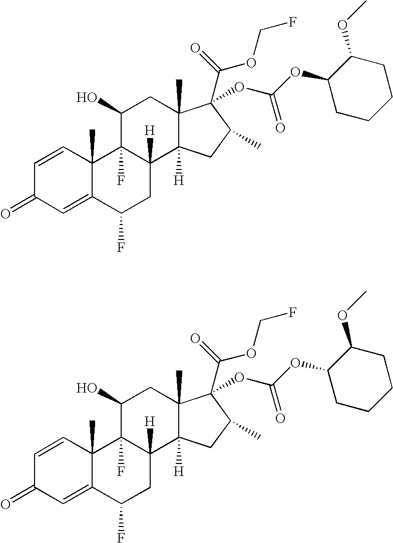

Example 18 was prepared as a ca 1:1 mixture of diastereomers from (6α,11β,16α,17α)-6,9-difluoro-11-hydroxy-16-methyl-17-[({[(1RS,2RS)-2-(methyloxy)cyclohexyl]oxy}carbonyl)oxy]-3-oxoandrosta-1,4-diene-17-carboxylic acid (Intermediate 18) using a method similar to that described for Example 11.

The diastereomers were then separated by reverse phase HPLC—isocratic conditions—eluting with 45% mobile phase B, run time 30 min, flow rate 20 ml/min.

Mobile phase A—water/0.1% formic acid v/v

Mobile phase B—95% aq Acetonitrile/0.05% formic acid v/v

### Example 18A

### LCMS retention time 3.51 min, m/z 585 MH⁺. ¹H-NMR: (DMSO-d₆, 400 MHz) 17β fluoromethylene protons δ 5.86 (d, 50 Hz) and δ 5.73 (d, 50 Hz)

### Example 18B

### LCMS retention time 3.56 min, m/z 585 MH⁺. ¹H-NMR: (DMSO-d₆, 400 MHz) 17β fluoromethylene protons δ 5.88 (d, 50.5 Hz) and δ 5.70 (d, 50.5 Hz)

### Example 19

### Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate

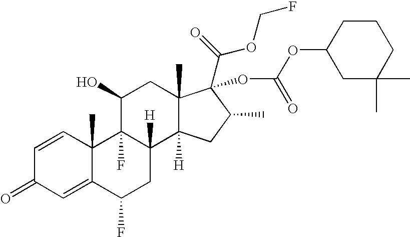

Sodium carbonate (289 mg, 2.72 mmol) was added to a stirred solution of (6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylic acid (Intermediate 19) (150 mg, 0.27 mmol) in anhydrous N,N-dimethylformamide (3 ml) and after stirring at room temperature for 15 minutes the mixture was cooled to −30° C. under nitrogen. Bromofluoromethane (41 μl, 0.73 mmol) was added and the reaction stirred at −25 to −35° C. for 2 hours. Further bromofluoromethane (41 μl, 0.73 mmol) was then added and the reaction stirred at −20 to −30° C. for 2.5 hours. The reaction was allowed to warm to room temperature, treated with diethylamine (79 μl, 1.2 mmol) and added dropwise to 5M hydrochloric acid. The resulting precipitate was filtered and dried in vacuo to give the title compound as a mixture of diastereomers:

The diastereomers were then separated using normal phase HPLC to give:

### Example 19A

### LCMS retention time 3.88 min, m/z 583 MH⁺. ¹H-NMR: (DMSO-d₆, 400 MHz) 17β fluoromethylene protons δ 5.87 (dd, 50.5, 2 Hz) and δ 5.76 (dd, 50.5, 2 Hz)

### Example 19B

### LCMS retention time 3.89 min, m/z 583 MH⁺. ¹H-NMR: (DMSO-d₆, 400 MHz) 17β fluoromethylene protons δ 5.86 (dd, 50, 2 Hz) and δ 5.74 (dd, 50, 2 Hz)

### Example 20

### Fluoromethyl(6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylate

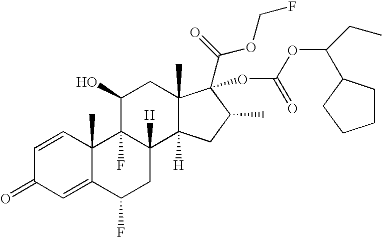

Example 20 was prepared as a mixture of diastereomers from (6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylic acid (Intermediate 20) using a method similar to that described for Example 4. The crude product was purified on a 5 g silica Bond Elut cartridge eluted using 0-100% diethylether in cyclohexane gradient over 30 minutes to give the title compound:

The diastereomers were then separated using normal phase HPLC to give:

### Example 20A

### LCMS retention time 3.82 min, m/z 583 MH⁺. ¹H-NMR: (DMSO-d₆, 400 MHz) 17β fluoromethylene protons δ 5.84 (d, 51 Hz) and δ 5.67 (d, 51 Hz)

### Example 20B

### LCMS retention time 3.84 min, m/z 583 MH⁺. ¹H-NMR: (DMSO-d₆, 400 MHz) 17β fluoromethylene protons δ 5.83 (d, 50.5 Hz) and δ 5.68 (d, 50.5 Hz)

### Example 21

### Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate

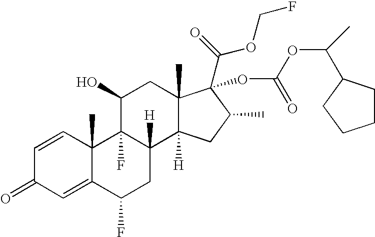

Example 21 was prepared as a mixture of diastereomers from (6α,11β,16α,17α)-17-({[(1-Cyclopentylethyl)oxy]carbonyl}oxy)-6,9-difluoro-11-hydroxy-16-methyl-3-oxoandrosta-1,4-diene-17-carboxylic acid (Intermediate 21) using a method similar to that described for Example 4. The crude product was purified on a 5 g silica Bond Elut cartridge eluted using 0-100% diethylether in cyclohexane gradient over 30 minutes to give the title compound:

The diastereomers were then separated using normal phase HPLC to give:

### Example 21A

### LCMS retention time 3.72 min, m/z 570 MH⁺. ¹H-NMR: (DMSO-d₆, 400 MHz) 17β fluoromethylene protons δ 5.85 (d, 50.5 Hz) and δ 5.70 (d, 50.5 Hz)

### Example 21B

### LCMS retention time 3.75 min, m/z 570 MH⁺. ¹H-NMR: (DMSO-d₆, 400 MHz) 17β fluoromethylene protons δ 5.85 (dd, 51, 1.5 Hz) and δ 5.75 (dd, 51, 1.5 Hz)

### Example 22

### Fluoromethyl(6α,11β,16α,17α)-6,9-difluoro-11-hydroxy-16-methyl-3-oxo-17-({[(1-propylbutyl)oxy]carbonyl}oxy)androsta-1,4-diene-17-carboxylate

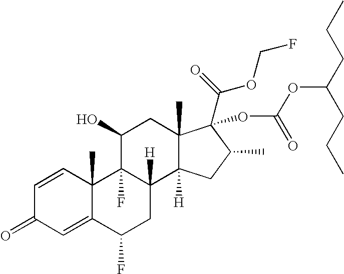

Example 22 was prepared from (6α,11β,16α,17α)-6,9-difluoro-11-hydroxy-16-methyl-3-oxo-17-({[(1-propylbutyl)oxy]carbonyl}oxy)androsta-1,4-diene-17-carboxylic acid (Intermediate 22) using a method similar to that described for Example 4. The crude product was purified on a 5 g silica Bond Elut cartridge using 1:1 diethylether cyclohexane to give the title compound: LCMS retention time 3.82 min, m/z 571 MH⁺

### Example 23

### Fluoromethyl(6α,11β,16α,17α)-6,9-difluoro-11-hydroxy-16-methyl-3-oxo-17-({[(1,2,2-trimethylpropyl)oxy]carbonyl}oxy)androsta-14-diene-17-carboxylate

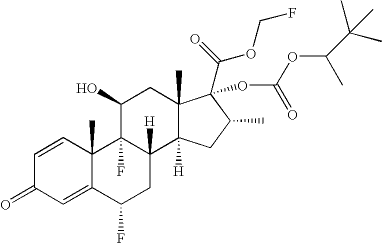

Example 23 was prepared as a mixture of diastereomers from (6α,11β,16α,17α)-6,9-Difluoro-11-hydroxy-16-methyl-3-oxo-17-({[(1,2,2-trimethylpropyl)oxy]carbonyl}oxy)androsta-1,4-diene-17-carboxylic acid (Intermediate 23) using a method similar to that described for Example 2. The crude product was purified on a 5 g silica Bond Elut cartridge using a 0-100% ethyl acetate in cyclohexane gradient over 60 minutes to give the title compound:

The diastereomers were then separated using normal phase HPLC to give:

### Example 23A

### LCMS retention time 3.77 min, m/z 557 MH⁺. ¹H-NMR: (DMSO-d₆, 400 MHz) 17β fluoromethylene protons δ 5.84 (dd, 50.5, 2 Hz) and δ 5.67 (dd, 50.5, 2 Hz)

### Example 23B

### LCMS retention time 3.78 min, m/z 557 MH⁺. ¹H-NMR: (DMSO-d₆, 400 MHz) 17β fluoromethylene protons δ 5.85 (dd, 50.5, 2 Hz) and δ 5.76 (dd, 50.5, 2 Hz)

### Example 24

### Fluoromethyl(6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylate

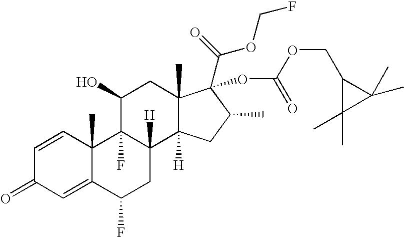

Example 24 was prepared from (6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylic acid (Intermediate 24) using a method similar to that described for Example 2. The crude product was purified on a 2 g silica Bond Elut cartridge using a 0-20% ethyl acetate in cyclohexane gradient to give the title compound: LCMS retention time 3.89 min, m/z 583 MH⁺

### Example 25

### Fluoromethyl(6α,11β,16α,17α)-6,9-difluoro-11-hydroxy-16-methyl-17-[({[1-(1-methylethyl)butyl]oxy}carbonyl)oxy]-3-oxoandrosta-1,4-diene-17-carboxylate

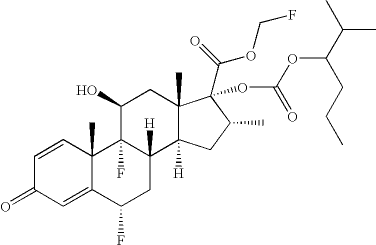

Example 25 was prepared as a mixture of diastereomers from (6α,11β,16α,17α)-6,9-difluoro-11-hydroxy-16-methyl-17-[({[1-(1-methylethyl)butyl]oxy}carbonyl)oxy]-3-oxoandrosta-1,4-diene-17-carboxylic acid (Intermediate 25) using a method similar to that described for Example 2. The crude product was purified on a 10 g silica Bond Elut cartridge using a 0-100% diethylether in cyclohexane gradient over 40 minutes to give the title compound: LCMS retention time 3.84 min, m/z 571 MH⁺

### Example 26

### Fluoromethyl(6α,11β,16α,17α)-6,9-difluoro-11-hydroxy-16-methyl-3-oxo-17-({[(2,2,3,3-tetramethylcyclopropyl)oxy]carbonyl}oxy)androsta-1,4-diene-17-carboxylate

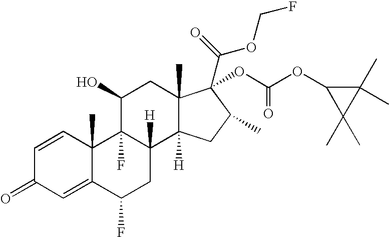

Example 26 was prepared from (6α,11β,16α,17α)-6,9-Difluoro-11-hydroxy-16-methyl-3-oxo-17-({[(2,2,3,3-tetramethylcyclopropyl)oxy]carbonyl}oxy)androsta-1,4-diene-17-carboxylic acid (Intermediate 26) using a method similar to that described for Example 8. The crude reaction mixture was applied to silica Bond Elut cartridges but this failed to give pure material. The crude reaction mixture was therefore purified by mass-directed autopreparation to give the title compound: LCMS retention time 3.82 min, m/z 569 MH⁺

### Example 27

### Fluoromethyl(6α,11β,16α,17α)-6,9-difluoro-11-hydroxy-16-methyl-3-oxo-17-[({[(1S,2R,4S)-1.7.7-trimethylbicyclo[2.2.1]hept-2-yl]oxy}carbonyl)oxy]androsta-1,4-diene-17-carboxylate

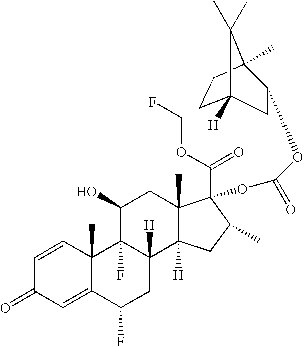

Example 27 was prepared from (6α,11β,16α,17α)-6,9-Difluoro-11-hydroxy-16-methyl-3-oxo-17-[({[(1S,2R,4S)-1,7,7-trimethylbicyclo[2.2.1]hept-2-yl]oxy}carbonyl)oxy]androsta-1,4-diene-17-carboxylic acid (Intermediate 27) using a method similar to that described for Example 10. The crude product was purified on a log silica Bond Elut cartridge using a 0-100% ethyl acetate in cyclohexane gradient over 20 minutes to give the title compound: LCMS retention time 3.95 min, m/z 609 MH⁺

### Example 28

### Fluoromethyl(6α,11β,16α,17α)-6,9-difluoro-11-hydroxy-16-methyl-3-oxo-17-[({[(1R,2S,4R)-1,7,7-trimethylbicyclo[2.2.1]hept-2-yl]oxy}carbonyl)oxy]androsta-1,4-diene-17-carboxylate

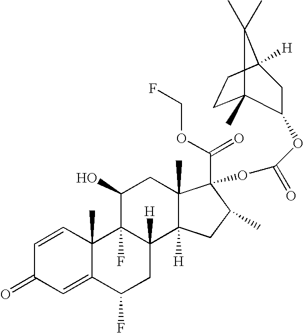

Example 28 was prepared from (6α,11β,16α,17α)-6,9-Difluoro-11-hydroxy-16-methyl-3-oxo-17-[({[(1R,2S,4R)-1,7,7-trimethylbicyclo[2.2.1]hept-2-yl]oxy}carbonyl)oxy]androsta-1,4-diene-17-carboxylic acid (Intermediate 28) using a method similar to that described for Example 10. The crude product was purified on a 10 g silica Bond Elut cartridge using a 0-100% ethyl acetate in cyclohexane gradient over 20 minutes to give the title compound: LCMS retention time 3.95 min, m/z 609 MH⁺

### Example 29

### Fluoromethyl(6α,11β,16α,17α)-6,9-difluoro-11-hydroxy-16-methyl-3-oxo-17-[({[(1R,2R,4R)-1,7,7-trimethylbicyclo[2.2.1]hept-2-yl]oxy}carbonyl)oxy]androsta-1,4-diene-17-carboxylate

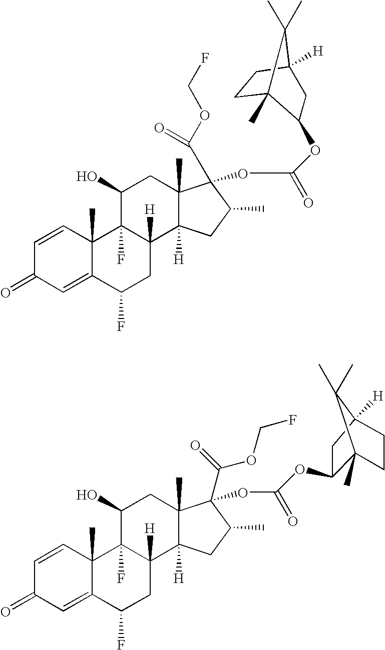

Example 29 was prepared as a mixture of diastereomers from (6α,11β,16α,17α)-6,9-difluoro-11-hydroxy-16-methyl-3-oxo-17-[({[(1RS,2RS,4RS)-1,7,7-trimethylbicyclo[2.2.1]hept-2-yl]oxy}carbonyl)oxy]androsta-1,4-diene-17-carboxylic acid (Intermediate 29) using a method similar to that described for Example 10. The diastereomers were then separated using normal phase HPLC to give:

### Example 29A

### LCMS retention time 4.00 min, m/z 609 MH⁺. ¹H-NMR: (DMSO-d₆, 400 MHz) 17β fluoromethylene protons δ 5.86 (dd, 50.5, 2 Hz) and δ 5.68 (dd, 50.5, 2 Hz)

### Example 29B

### LCMS retention time 4.00 min, m/z 609 MH⁺. ¹H-NMR: (DMSO-d₆, 400 MHz) 17β fluoromethylene protons δ 5.84 (dd, 50.5, 2 Hz) and δ 5.74 (dd, 50.5, 2 Hz)

### Example 30

### Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate

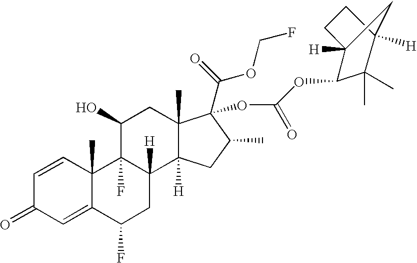

Example 30 was prepared from (6a,11,B,16a,17a)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylic acid (Intermediate 30) using a method similar to that described for Example 4. The crude product was purified on a 10 g silica Bond Elut cartridge using a 0-100% diethylether in cyclohexane gradient over 40 minutes to give the title compound: LCMS retention time 3.88 min, m/z 595 MH⁺

### Example 31

### Fluoromethyl(6α,11β,16α,17α)-17-{[(cyclopentyloxy)carbonyl]oxy}-6,9-difluoro-11-hydroxy-16-methyl-3-oxoandrosta-1,4-diene-17-carboxylate

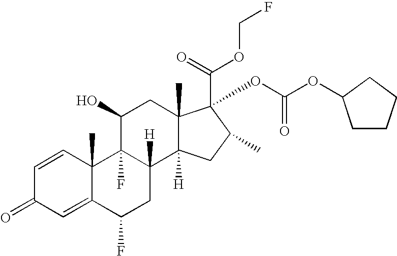

Sodium carbonate (313 mg, 2.95 mmol) was added to a stirred solution of (6α,11β,16α,17α)-17-{[(cyclopentyloxy)carbonyl]oxy}-6,9-difluoro-11-hydroxy-16-methyl-3-oxoandrosta-1,4-diene-17-carboxylic acid (Intermediate 31) (150 mg, 0.29 mmol) in anhydrous N,N-dimethylformamide (5 ml) and after stirring at room temperature for 30 minutes the mixture was cooled to −20° C. Bromofluoromethane (45 μl, 0.80 mmol) was added and the reaction stirred at −20° C. for 3 hours followed by overnight at room temperature. The reaction was then treated with diethylamine (39 μl, 0.38 mmol), 2M hydrochloric acid (10 ml) and water (10 ml). The product was extracted into dichloromethane (10 ml) which was separated, washed with saturated aqueous sodium hydrogen carbonate solution (10 ml) followed by brine/water and evaporated in vacuo. The crude product was purified on a 10 g silica Bond Elut cartridge eluted using a 10-40% ethyl acetate in cyclohexane gradient to give the title compound (125 mg): LCMS retention time 3.63 min, m/z 541 MH⁺

### Example 32

### Fluoromethyl(6α,11β,16α,17α)-6,9-difluoro-11-hydroxy-16-methyl-17-[({[(1 S,2R,5S)-5-methyl-2-(1-methylethyl)cyclohexyl]oxy}carbonyl)oxy]-3-oxoandrosta-1,4-diene-17-carboxylate

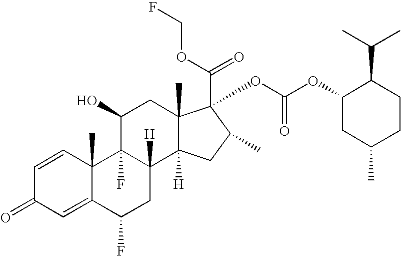

Example 32 was prepared from (6α,11β,16α,17α)-6,9-difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylic acid (Intermediate 32) using a method similar to that described for Example 1. LCMS retention time 4.08 min, m/z 611 MH⁺

### Example 33

### Fluoromethyl(6α,11β,16α,17α)-6,9-difluoro-11-hydroxy-16-methyl-3-oxo-17-[({[(1R,2R,4S)-1,3,3-trimethylbicyclo[2.2.1]hept-2-yl]oxy}carbonyl)oxy]androsta-1,4-diene-17-carboxylate

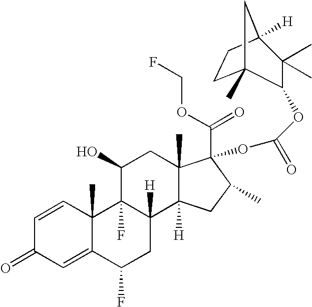

Example 33 was prepared from (6α,11β,16α,17α)-6,9-difluoro-11-hydroxy-16-methyl-3-oxo-17-[({[(1R,2R,4S)-1,3,3-trimethylbicyclo[2.2.1]hept-2-yl]oxy}carbonyl)oxy]androsta-1,4-diene-17-carboxylic acid (Intermediate 33) using a method similar to that described for Example 10. The crude product was purified on a 10 g silica Bond Elut cartridge using a 0-100% ethyl acetate in cyclohexane gradient over 20 minutes to give the title compound: LCMS retention time 4.00 min, m/z 609 MH⁺

### Example 34

### Fluoromethyl(6α,11β,16α,17α)-6,9-difluoro-11-hydroxy-16-methyl-3-oxo-17-[({[(1R,2R,3R,5S)-2,6,6-trimethylbicyclo[3.1.1]hept-3-yl]oxy}carbonyl)oxy]androsta-1,4-diene-17-carboxylate

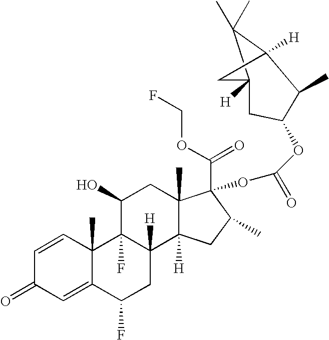

Example 34 was prepared from (6α,11β,16α,17α)-6,9-Difluoro-11-hydroxy-16-methyl-3-oxo-17-[({[(1R,2R,3R,5S)-2,6,6-trimethylbicyclo[3.1.1]hept-3-yl]oxy}carbonyl)oxy]androsta-1,4-diene-17-carboxylic acid (Intermediate 34) using a method similar to that described for Example 10. The crude product was purified on a 10 g silica Bond Elut cartridge using a 0-100% ethyl acetate in cyclohexane gradient over 20 minutes to give the title compound: LCMS retention time 4.00 min, m/z 609 MH⁺

### Example 35

### Fluoromethyl(6α,11β,16α,17α)-6,9-difluoro-11-hydroxy-16-methyl-3-oxo-17-[({[(1S,2S,3S,5R)-2,6,6-trimethylbicyclo[3.1.1]hent-3-yl]oxy}carbonyl)oxy]androsta-1,4-diene-17-carboxylate

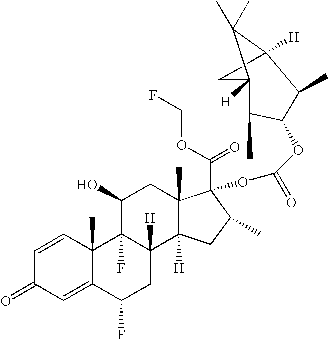

Example 35 was prepared from (6α,11β,16α,17α)-6,9-Difluoro-11-hydroxy-16-methyl-3-oxo-17-[({[(1S,2S,3S,5R)-2,6,6-trimethylbicyclo[3.1.1]hept-3-yl]oxy}carbonyl)oxy]androsta-1,4-diene-17-carboxylic acid (Intermediate 35) using a method similar to that described for Example 10. The crude product was purified on a 10 g silica Bond Elut cartridge using a 0-100% ethyl acetate in cyclohexane gradient over 20 minutes to give the title compound: LCMS retention time 4.00 min, m/z 609 MH⁺

### Example 36

### Fluoromethyl(6α,11β,16α,17α)-17-({[(cis-4-ethylcyclohexyl)oxy]carbonyl}oxy)-6,9-difluoro-11-hydroxy-16-methyl-3-oxoandrosta-1,4-diene-17-carboxylate

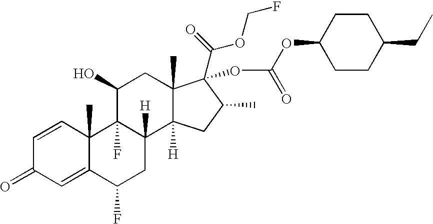

Example 36 was prepared from (6α,11β,16α,17α)-17-({[(cis-4-ethylcyclohexyl)oxy]carbonyl}oxy)-6,9-difluoro-11-hydroxy-16-methyl-3-oxoandrosta-1,4-diene-17-carboxylic acid (Intermediate 36) using a method similar to that described for Example 19. The crude product was purified on a 1 g silica Bond Elut cartridge eluting with 1:1 diethylether:cyclohexane to give the title compound: LCMS retention time 3.92 min, m/z 583 MH⁺

### Example 37

### Fluoromethyl(6α,11β,16α,17α)-17-({[(trans-4-ethylcyclohexyl)oxy]carbonyl}oxy)-6,9-difluoro-11-hydroxy-16-methyl-3-oxoandrosta-1,4-diene-17-carboxylate

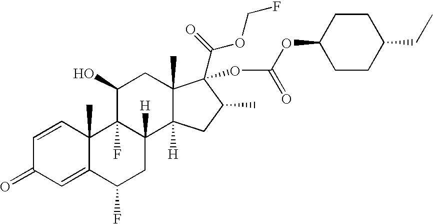

Example 37 was prepared from (6α,11β,16α,17α)-17-({[(trans-4-ethylcyclohexyl)oxy]carbonyl}oxy)-6,9-difluoro-11-hydroxy-16-methyl-3-oxoandrosta-1,4-diene-17-carboxylic acid (Intermediate 37) using a method similar to that described for Example 19. The crude product was purified on a 1 g silica Bond Elut cartridge eluting with 1:1 diethylether:cyclohexane to give the title compound: LCMS retention time 3.97 min, m/z 583 MH⁺

### Example 38

### Fluoromethyl(6α,11β,16α,17α)-17-({[(1-ethyl-2,2-dimethylpropyl)oxy]carbonyl}oxy)-6,9-difluoro-11-hydroxy-16-methyl-3-oxoandrosta-1,4-diene-17-carboxylate

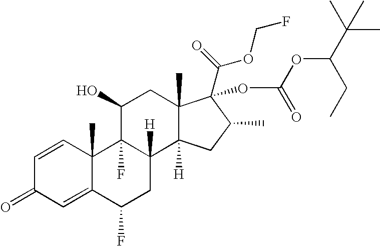

Example 38 was prepared as a mixture of diastereomers from (6α,11β,16α,17α)-17-({[(1-Ethyl-2,2-dimethylpropyl)oxy]carbonyl}oxy)-6,9-difluoro-11-hydroxy-16-methyl-3-oxoandrosta-1,4-diene-17-carboxylic acid (Intermediate 38) using a method similar to that described for Example 2. The crude product was purified on a 10 g silica Bond Elut cartridge eluted using a 0-100% diethylether in cyclohexane gradient over 40 minutes to give the title compound: LCMS retention time 3.82 min, m/z 571 MH⁺

### Pharmacological Activity

Pharmacological activity may be assessed in functional in vitro assays of glucocorticoid agonist activity.

### Assay for Transrepression Activity of the Glucocorticoid Agonists

The functional assay based on that described by K. P. Ray et al., Biochem J. (1997), 328, 707-715 provides a measure of transrepressive activity of a glucocorticoid agonist. A549 cells stably transfected with a reporter gene containing the NF-κB responsive elements from the ELAM gene promoter coupled to sPAP (secreted alkaline phosphatase) are treated with test compounds at appropriate doses for 1 hour at 37° C. The cells are then stimulated with tumour necrosis factor (TNF, 10 ng/ml) for 16 hours, at which time the amount of alkaline phosphatase produced is measured by a standard colourimetric assay. Dose response curves were constructed from which EC₅₀ values were estimated.

The pIC₅₀ values for compounds of Examples 1 to 38 were >8.0 in this assay.

The pIC₅₀ values for compounds of Examples 1 to 11A, 12A to 20A, 21A to 23A, 24 to 31 and 33 to 38 were >9.0 in this assay.

The pIC₅₀ values of Examples 1, 2, 6, 8, 9, 12B, 13, 15, 18A, 18B 19A, 21A, 23A and 30 were >10 in this assay.

### Assay for Transactivation Activity of the Glucocorticoid Agonists

The functional assay based on that described by R. J. H. Austin et al., Eur Resp J. (2002), 20, 1386-1392 measures the ability of compounds to directly transactivate gene expression. A549 cells stably transfected with a reporter gene containing the glucocorticoid responsive region of the mouse mammary tumour virus long terminal repeat (MMTV-LTR) coupled to renilla luciferase were treated with test compounds at appropriate doses for 6 hour at 37° C. The amount of luciferase activity present within the cells is then determined by measuring the light emitted following incubation with a suitable substrate. Dose response curves were constructed from which EC₅₀ values were estimated and from which maximal responses are calculated relative to Dexamethasone (100%).

Compounds of Examples 1 to 38 showed maximal responses of <25% in this assay.

Compounds of Examples 2, 3, 5, 7 to 13, 19A to 25, 27 to 30 and 32 to 38 showed maximal responses of <10% in this assay.

### Assay for Progesterone Receptor Activity

A T225 flask of CV-1 cells at a density of 80% confluency was washed with PBS, detached from the flask using 0.25% trypsin and counted using a Sysmex KX-21N. Cells were diluted in DMEM containing 10% Hyclone, 2 mM L-Glutamate and 1% Pen/Strep at 140 cells/μl and transduced with 10% PRb-BacMam and 10% MMTV-BacMam. 70 ml of suspension cells were dispensed to each well of white Nunc 384-well plates, containing compounds at the required concentration. After 24 h 10 μl of Steady Glo were added to each well of the plates. Plates were incubated in the dark for 10 min before reading them on a Viewlux reader. Dose response curves were constructed from which pEC₅₀ values were estimated.

The pEC₅₀ values for compounds of Examples 2, 4 to 6, 8, 10A to 11B, 14, 18A, 18B, 20A to 23B, 25 to 28, 29B, 30, 32, 34 and 38 were <8 in this assay.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

The patents and patent applications described in this application are herein incorporated by reference.

What is claimed is:

1. A compound of formula (I): 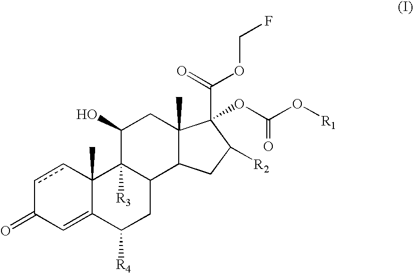
wherein
R₁ represents C₄-C₇ branched chain alkyl,
C₃-C₈ cycloalkyl optionally substituted by one or more groups independently selected from C₁-C₃ alkyl and methoxy,
C₄-C₆ cycloalkylmethyl wherein the methyl group is optionally substituted by a group selected from methyl or ethyl,
or a bicycloalkyl group optionally substituted by one or more methyl groups;
R₂ represents hydrogen, a methyl group, which may be in either the α or β configuration, or a methylene group;
R₃ and R₄ are the same or a different group and each independently represents hydrogen, halogen or a methyl group;
and [symbol] represents a single or a double bond;
or a physiologically acceptable solvate thereof.

2. A compound as claimed in claim 1 wherein R₁ represents C₄-C₆ branched chain alkyl.

3. A compound as claimed in claim 2 wherein R₁ is selected from the group consisting of 1,1-dimethylethyl, 1,1-dimethylpropyl, 2-ethylbutyl, 1-ethyl-2-methylpropyl, 1,2-dimethylpropyl and a 1,2,2-trimethylpropyl Isomer A group.

4. A compound as claimed in claim 1 wherein R₁ represents cyclohexyl optionally substituted by one or more groups independently selected from C₁-C₃ alkyl and methoxy.

5. A compound as claimed in claim 4 wherein R₁ represents cyclohexyl optionally substituted by one or more groups independently selected from methyl and methoxy.

6. A compound as claimed in claim 4 wherein R₁ represents (1R,2R)-2-(methyloxy)cyclohexyl, (1S,2S)-2-(methyloxy)cyclohexyl or a 3,3-dimethylcyclohexyl Isomer A group.

7. A compound as claimed in claim 1 wherein R₁ represents cyclopentylmethyl wherein the methyl group is optionally substituted by a group selected from methyl or ethyl.

8. A compound as claimed in claim 7 wherein R₁ represents cyclopentylmethyl or a 1-cyclopentylethyl Isomer A group.

9. A compound as claimed in claim 1 wherein R₁ represents a bicycloalkyl group optionally substituted by one or more methyl groups.

10. A compound as claimed in claim 9 wherein R₁ is selected from the group consisting of 1 RS,2RS,4SR-bicyclo[2.2.1]hept-2-yl Isomer B, 1 RS,2SR,4SR bicyclo[2.2.1]hept-2-yl or and (1R,2R,4S)-3,3-dimethylbicyclo{2.2.1]hept-2-yl group.

11. A compound as claimed in claim 1 wherein R₂ represents a methyl group in the α-configuration.

12. A compound as claimed in claim 1 wherein R₃ and R₄ are both fluorine.

13. A compound as claimed in claim 1 wherein [symbol] represents a double bond.

14. A compound which is selected from the group consisting of
Fluoromethyl(6α,11β,16α,17α)-17-({[(1,1-dimethylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylate;
Fluoromethyl(6α,11β,16α,17α)-17-({[(1-ethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[2-methyl-1-(1-methylethyl)propyl]oxy}carbonyl)oxy]-3-oxoandrosta-1,4-diene-17-carboxylate;
Fluoromethyl(6α,11β,16α,17α)-17-({[(2-ethylbutyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(2,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1S,2R)-2-methylcyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1R,2S)-2-methylcyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[4-(1-methylethyl)cyclohexyl]oxy}carbonyl)oxy]-3-oxoandrosta-1,4-diene-17-carboxylate;
Fluoromethyl(6α,11β,16α,17α)-17-({[(1S,2S,4R)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1R,2R,4S)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-{[(cycloheptyloxy)carbonyl]oxy}-6,9-difluoro-11-hydroxy-16-methyl-3-oxoandrosta-1,4-diene-17-carboxylate;
Fluoromethyl(6α,11β,16α, 17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-{[(cyclooctyloxy)carbonyl]oxy}-6,9-difluoro-11-hydroxy-16-methyl-3-oxoandrosta-1,4-diene-17-carboxylate;
Fluoromethyl(6α,11β,16α,17α)-17-[({[(1S,3R,5S)-3,5-dimethylcyclohexyl]oxy}carbonyl)oxy]-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-3-oxo-17-({[(1-propylbutyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylate;
Fluoromethyl(6α,11β,16α,17α)-6,9-difluoro-11-hydroxy-16-methyl-17-[({[1-(1-methylethyl)butyl]oxy}carbonyl)oxy]-3-oxoandrosta-1,4-diene-17-carboxylate;
Fluoromethyl(6α,11β,16α,17α)-6,9-difluoro-11-hydroxy-16-methyl-3-oxo-17-({[(2,2,3,3-tetramethylcyclopropyl)oxy]carbonyl}oxy)androsta-1,4-diene-17-carboxylate;
Fluoromethyl(6α,11β,16α,17α)-6,9-difluoro-11-hydroxy-16-methyl-3-oxo-17-[({[(1S,2R,4S)-1,7,7-trimethylbicyclo[2.2.1]hept-2-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1R,2S,4R)-1,7,7-trimethylbicyclo[2.2.1]hept-2-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1R,2R,4R)-1,7,7-trimethylbicyclo[2.2.1]hept-2-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1S,2S,4S)-1,7,7-trimethylbicyclo[2.2.1]hept-2-yl]oxy}carbonyl)oxy]androsta-1,4-diene-17-carboxylate;
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate;
Fluoromethyl(6α,11β,16α,17α)-17-{[(cyclopentyloxy)carbonyl]oxy}-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-3-oxo-17-[({[(1R,2R,4S)-1,3,3-trimethylbicyclo[2.2.1]hept-2-yl]oxy}carbonyl)oxy]androsta-1,4-diene-17-carboxylate
Fluoromethyl(6α,11β,16α,17α)-6,9-difluoro-11-hydroxy-16-methyl-3-oxo-17-[({[(1R,2R,3R,5S)-2,6,6-trimethylbicyclo[3.1.1]hept-3-yl]oxy}carbonyl)oxy]androsta-1,4-diene-17-carboxylate;
Fluoromethyl(6α,11β,16α,17α)-6,9-difluoro-11-hydroxy-16-methyl-3-oxo-17-[({[(1S,2S,3S,5R)-2,6,6-trimethylbicyclo[3.1.1]hept-3-yl]oxy}carbonyl)oxy]androsta-1,4-diene-17-carboxylate;
Fluoromethyl(6α,11β,16α,17α)-17-({[(cis-4-ethylcyclohexyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(trans-4-ethylcyclohexyl)oxy]carbonyl}oxy)-6,9-difluoro-11-hydroxy-16-methyl-3-oxoandrosta-1,4-diene-17-carboxylate; or
Fluoromethyl(6α,11β,16α,17α)-17-({[(1-ethyl-2,2-dimethyl propyl)oxy]carbonyl}oxy)-6,9-difluoro-11-hydroxy-16-methyl-3-oxoandrosta-1,4-diene-17-carboxylate.

15. A compound as claimed in claim 14 which is selected from the group consisting of
Fluoromethyl(6α,11β,16α,17α)-17-({[(1,1-dimethylethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(2-ethylbutyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylate Isomer B;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate Isomer A; or
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate.

16. A compound as claimed in claim 15 which is selected from the group consisting of
Fluoromethyl(6α,11β,16α,17α)-17-({[(1,1-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1-ethyl-2-methylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1,2-dimethylpropyl)oxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(1RS,2RS,4SR)-bicyclo[2.2.1]hept-2-yloxy]carbonyl}oxy)-6,9-difluoro-11-hydroxy-16-methyl-3-oxoandrosta-1,4-diene-17-carboxylate Isomer B;
Fluoromethyl(6α,11β,16α,17α)-17-({[(1RS,2SR,4SR)-bicyclo[2.2.1]hept-2-yloxy]carbonyl}oxy)-6,9-difluoro-11-hydroxy-16-methyl-3-oxoandrosta-1,4-diene-17-carboxylate;
Fluoromethyl(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylate;
Fluoromethyl(6α,11β,16α,17α)-6,9-difluoro-11-hydroxy-16-methyl-17-[({[(1R,2R)-2-(methyloxy)cyclohexyl]oxy}carbonyl)oxy]-3-oxoandrosta-1,4-diene-17-carboxylate;
Fluoromethyl(6α,11β,16α,17α)-6,9-difluoro-11-hydroxy-16-methyl-17-[({[(1S,2S)-2-(methyloxy)cyclohexyl]oxy}carbonyl)oxy]-3-oxoandrosta-1,4-diene-17-carboxylate;
Fluoromethyl(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylate Isomer A;
Fluoromethyl(6α,11β,16α,17α)-6,9-difluoro-11-hydroxy-16-methyl-3-oxo-17-({[(1,2,2-trimethylpropyl)oxy]carbonyl}oxy)androsta-1,4-diene-17-carboxylate Isomer A; or
Fluoromethyl(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylate.

17-18. (canceled)

19. A pharmaceutical composition comprising a compound of formula (I), or a physiologically acceptable solvate thereof, as defined in claim 1 together, if desirable, in admixture with one or more physiologically acceptable diluents or carriers.

20. A pharmaceutical composition as claimed in claim 19 which is an aerosol formulation further comprising a fluorocarbon or hydrogen-containing chlorofluoro carbon as propellant, optionally in combination with a surfactant and/or a cosolvent.

21. A pharmaceutical composition according to claim 19 which further comprises a therapeutically active agent.

22. A pharmaceutical composition according to claim 21 in which said therapeutically active agent is a β₂-adrenoreceptor agonist.

23. A method for the treatment of a human or animal subject with an anti-inflammatory and/or allergic condition, which method comprises administering to said human or animal subject an effective amount of a compound of formula (I) as defined in claim 1 or a physiologically acceptable solvate thereof.

24. A compound which is selected from the group consisting of:
(6α,11β,16α,17α)-6,9-difluoro-11-hydroxy-16-methyl-17-[({[(1R,2S,5R)-5-methyl-2-(1-methylethyl)cyclohexyl]oxy}carbonyl)oxy]-3-oxoandrosta-1,4-diene-17-carboxylic acid;
(6α,11β,16α,17α)-17-{[(cycloheptyloxy)carbonyl]oxy}-6,9-difluoro-11-hydroxy-16-methyl-3-oxoandrosta-1,4-diene-17-carboxylic acid;
(6α,11β,16α,17α)-17-({[(cyclopentylmethyl)oxy]carbonyl}oxy)-6,9-difluoro-11-hydroxy-16-methyl-3-oxoandrosta-1,4-diene-17-carboxylic acid;
(6α,11β,16α,17α)-17-{[(cyclooctyloxy)carbonyl]oxy}-6,9-difluoro-11-hydroxy-16-methyl-3-oxoandrosta-1,4-diene-17-carboxylic acid;
(6α,11β,16α,17α)-17-[({[(1S,3R,5S)-3,5-dimethylcyclohexyl]oxy}carbonyl)oxy]-6,9-difluoro-11-hydroxy-16-methyl-3-oxoandrosta-1,4-diene-17-carboxylic acid;
(6α,11β,16α,17α)-6,9-difluoro-11-hydroxy-16-methyl-17-[({[(1RS,2RS)-2-(methyloxy)cyclohexyl]oxy}carbonyl)oxy]-3-oxoandrosta-1,4-diene-17-carboxylic acid;
(6α,11β,16α,17α)-17-({[(3,3-dimethylcyclohexyl)oxy]carbonyl}oxy)-6,9-difluoro-11-hydroxy-16-methyl-3-oxoandrosta-1,4-diene-17-carboxylic acid;
(6α,11β,16α,17α)-17-({[(1-cyclopentylpropyl)oxy]carbonyl}oxy)-6,9-difluoro-11-hydroxy-16-methyl-3-oxoandrosta-1,4-diene-17-carboxylic acid;
(6α,11β,16α,17α)-17-({[(1-cyclopentylethyl)oxy]carbonyl}oxy)-6,9-difluoro-11-hydroxy-16-methyl-3-oxoandrosta-1,4-diene-17-carboxylic acid;
(6α,11β,16α,17α)-6,9-difluoro-11-hydroxy-16-methyl-3-oxo-17-[({[(2,2,3,3-tetramethylcyclopropyl)methyl]oxy}carbonyl)oxy]androsta-1,4-diene-17-carboxylic acid;
(6α,11β,16α,17α)-6,9-difluoro-11-hydroxy-16-methyl-3-oxo-17-({[(2,2,3,3-tetramethylcyclopropyl)oxy]carbonyl}oxy)androsta-1,4-diene-17-carboxylic acid;
(6α,11β,16α,17α)-17-[({[(1R,2R,4S)-3,3-dimethylbicyclo[2.2.1]hept-2-yl]oxy}carbonyl)oxy]-6,9-difluoro-11-hydroxy-16-methyl-3-oxoandrosta-1,4-diene-17-carboxylic acid;
(6α,11β,16α,17α)-6,9-difluoro-11-hydroxy-16-methyl-17-[({[(1S,2R,5S)-5-methyl-2-(1-methylethyl)cyclohexyl]oxy}carbonyl)oxy]-3-oxoandrosta-1,4-diene-17-carboxylic acid; sand
(6α,11β,16α,17α)-17-({[(1-ethyl-2,2-dimethylpropyl)oxy]carbonyl}oxy)-6,9-difluoro-11-hydroxy-16-methyl-3-oxoandrosta-1,4-diene-17-carboxylic acid.

25. A process for preparing a compound of formula (I) as claimed in claim 1, which comprises reacting a carboxylic acid of formula (II); 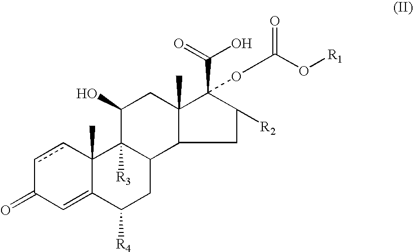
wherein R₁, R₂, R₃, R₄ and [symbol] are as defined above,
with a compound of formula L-CH₂—F wherein L represents a leaving group.

26. A process for preparing a compound of formula (II) 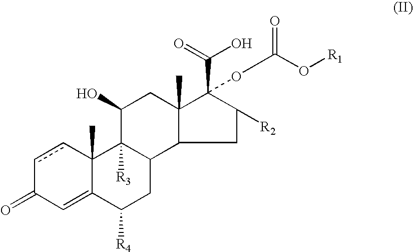
wherein
R₁ represents C₄-C₇ branched chain alkyl,
C₃-C₈ cycloalkyl optionally substituted by one or more groups independently selected from C₁-C₃ alkyl and methoxy,
C₄-C₆ cycloalkylmethyl wherein the methyl group is optionally substituted by a group selected from methyl or ethyl,
or a bicycloalkyl group optionally substituted by one or more methyl groups;
R₂ represents hydrogen, a methyl group, which may be in either the α or β configuration, or a methylene group;
R₃ and R₄ are the same or a different group and each independently represents hydrogen, halogen or a methyl group;
and [symbol] represents a single or a double bond;
which process comprises reacting a hydroxyacid (III) 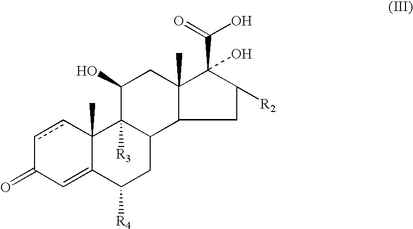
with a chloroformate R₁OCOCl or anhydride (R₁OCO)₂O in pyridine solution; wherein R₂, R₃, R₄ and [symbol] are as defined above.

27. The method according to claim 23, which method comprises administering to a human subject an effective amount of the compound of formula (I) or a physiologically acceptable solvate thereof.
